(19) United States
(12) Patent Application Publication
(10) Pub. No.: US 2011/0224343 A1
(43) Pub. Date: Sep. 15, 2011

(54) MODIFIER FOR AROMATIC POLYESTER AND AROMATIC POLYESTER RESIN COMPOSITION COMPRISING THE SAME

(76) Inventors: Masayoshi TABATA, Muroran-shi (JP); Takayoshi YAMAZAKI, Yamatokoriyama-shi (JP)

(21) Appl. No.: 12/871,363

(22) Filed: Aug. 30, 2010

(30) Foreign Application Priority Data
Mar. 13, 2010 (JP) 2010-56820

(51) Int. Cl. C08K 5/12; C07C 69/76

(52) U.S. Cl. 524/296; 560/76

(57) ABSTRACT

The present invention provides a modifier for aromatic polyesters which enhances the melt fluidity of aromatic polyesters without a significant decrease in the heat resistance of the aromatic polyesters, and an aromatic polyester resin composition including the modifier for aromatic polyesters. The present invention relates to a modifier for aromatic polyesters comprising polyhydric phenol residues and residues of aromatic polycarboxylic acid, acid halide or acid anhydride thereof, and the modifier comprises a material having a structure composed of a first residue selected from the group consisting of divalent residues represented by Formula (I): —Ar—W¹ₓ—Ar— and by Formula (II): —Ar—, the first residues being bonded to two identical or different second residues selected from the group consisting of monovalent residues represented by Formula (III):

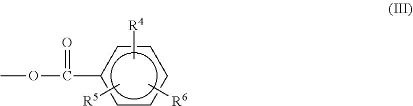

and monovalent residues represented by Formula (IV): —O—C(O)—R⁷—.

### BACKGROUND OF THE INVENTION

The present invention relates to a modifier for aromatic polyesters, and an aromatic polyester resin composition comprising the modifier, and more particularly a flow modifier for aromatic polyesters and an aromatic polyester resin composition comprising the modifier.

Aromatic polyesters derived from polyhydric phenols, e.g., bisphenol A, and aromatic polycarboxylic acids, acid halides or acid anhydrides thereof, e.g., isophthaloyl or terephthalic dichloride, usually have a high glass transition temperature, and high heat resistance. The aromatic polyesters, however, generally have such low fluidity whose polymers need to be heated to a temperature of 300° C. or higher during processing such as injection molding. Accordingly, the molded product is colored slightly yellow and unsuitable for optical applications such as optical fibers.

Attempts have been made to enhance the melt fluidity of resins including aromatic polyesters. For example, Japanese Patent Application Laid-Open No. 2007-113011 discloses a resin composition including a resin component containing a specified amount of at least one resin selected from the group consisting of polyphenylene ether resins, polycarbonate resins, polysulfone resins, polyether sulfone resins, polyarylate resins, polyamide-imide resins, polyether-imide resins, and thermoplastic polyimide resins, and a specified amount of an organic compound having a melting point of 200° C. or higher. This invention aims at compatibility of enhanced fluidity of the resin composition and high heat-resistance by blending the organic compound. The organic compound to be used is a phenolic stabilizer. In the examples in the specification of the Japanese Patent Application, polyphenylene ether resins and polycarbonate resins are used as resin components. Japanese Patent Application Laid-Open No. 2009-40840 discloses a thermoplastic polyester resin composition including a specified amount of a thermoplastic polyester resin and a specified amount of a thermosetting resin having a higher thermal deformation temperature than that of the thermoplastic polyester resin, and states that the thermoplastic polyester resin may be an aromatic polyester. This invention aims at rendering a thermoplastic polyester resin composition fluidable by the thermoplastic polyester resin containing a specified thermosetting resin. The disclosed thermosetting polyester resins are phenolic resins and melamine resins. Japanese Patent Application Laid-Open No. 2009-197057 discloses a resin molding material including a linear thermoplastic polymer composed mainly of at least one selected from the group consisting of polycarbonates, polyarylates, polyalkylene naphthalates and polyphenylene ethers; a specified phyllosilicate; and a macrocyclic oligomer as an additive to the polymer, in a specific proportion. In this invention, a concentrate of a macrocyclic oligomer containing a phyllosilicate is blended with the linear thermoplastic polymer to improve fluidity, resulting in a resin molded article having excellent thin wall moldability and rigidity. Japanese Patent Application Laid-Open No. 2008-222996 discloses a resin composition including a cyclic polyphenylene sulfide compound having a specific structure compounded in an amorphous resin. The disclosed amorphous resins are amorphous nylon resins, polycarbonate resins, polyarylate resins, ABS resins, poly(meth) acrylate resins and poly(meth)acrylate copolymers. In this invention, a cyclic polyphenylene sulfide compound having a specific structure is compounded to an amorphous resin to impart high fluidity to the resin. Japanese Patent Application Laid-Open No. 2009-132851 discloses a thermoplastic resin composition including a polyester resin, an amorphous resin, a flow modifier, and a stabilizer in a specific proportion. The disclosed amorphous resin in this invention is polyarylate. The flow modifier is a compound that has three or more functional groups selected from a hydroxyl group, a carboxyl group, an amino group, a glycidyl group, an isocyanate group, an ester group, or an amide group. This invention aims at compatibility of enhanced fluidity and satisfactory mechanical characteristics by blending the flow modifier. Japanese Patent Application Laid-Open No. 2009-132896 discloses a thermoplastic resin composition including a blend of a specified amount of at least one amorphous resin (a) selected from polycarbonate resins, polyphenylene ether resins, polyetherimide resins, polyamide imide resins, polysulfone resins, polyethersulfone resins, polyarylate resins, amorphous polyamide resins and polyphenylene sulfide sulfone resins; a specified amount of thermoplastic resin (b) other than the amorphous resin (a); and a dendritic polyester resin including at least one structural unit selected from an aromatic oxycarbonyl unit, an aromatic and/or aliphatic dioxy unit, and an aromatic dicarbonyl unit, and a tri- or more functional organic residue, the content of the functional organic residue being in a specified range on the basis of the total monomer units constituting the dendritic polyester. This invention aims at improving fluidity by blending the complicated dendritic polyester into the thermoplastic resin composition. All the inventions described above involve addition of the third substances, e.g., a flow modifier, to polyesters, e.g., aromatic polyesters, to enhance the flowability of the overall polyester resin composition. The resin composition can be thereby molded at a lower temperature without coloration. Unfortunately, the addition of the third substance sometimes inversely affects other characteristics regardless of enhanced fluidity, and the use of the special third substance may lead to an increase in cost.

Meanwhile, attempts have been made to protect the end moieties of an aromatic polyester to prevent coloration during molding processes. For example, Japanese Patent Application Laid-Open No. 2007-320989 discloses a method for preparing a terminal-protected polyester having a specified molecular weight by the reaction of an aromatic polyhydric alcohol with an aromatic polycarboxylic acid or its acid halide or acid anhydride, in the presence of a compound represented by X—C(O)—R, for example, benzoyl chloride during the reaction. The aromatic polyester prepared by the method gives a molded product that is barely colored despite being heated to a higher temperature to increase fluidity for molding. Accordingly, the aromatic polyester is suitable for optical products such as optical fibers.

### SUMMARY OF THE INVENTION

It is an object of the present invention to provide a modifier for aromatic polyesters and an aromatic polyester resin composition comprising the modifier for aromatic polyesters, which can enhance the melt fluidity of aromatic polyesters without a significant decrease in the heat resistance of the aromatic polyesters.

An aromatic polyester produced by the process described in Japanese Patent Application Laid-Open No. 2007-320989 has high transparency as well as high resistance, and is barely colored during the processing at a high temperature. The barely colored product, however, is not sufficiently transparent for optical applications. Accordingly, in order to prepare an aromatic polyester with much higher transparency, the inventors of the present invention made intensive and extensive investigations with the view that the addition of the flow modifier to a terminal-protected aromatic polyester such as those of Japanese Patent Application Laid-Open No. 2007-320989 could allow the aromatic polyester to be molded at a lower temperature, leading to a far more transparent molded article of the aromatic polyester. Unfortunately, although the conventional flow modifier improved the fluidity of the aromatic polyesters, it caused coloration of the molded article and inversely affected other characteristics, for example, decreasing heat resistance significantly. The inventors of the present invention have conducted the studies on flow modifiers that can improve the fluidity of the aromatic polyesters without the adverse effects. As a result, the inventors have found that the use of the specific flow modifiers described bellow can solve all the problems. Thus, the present invention has been completed.

Accordingly, the present invention provides:

(1) A modifier for aromatic polyesters comprising polyhydric phenol residues and residues of aromatic polycarboxylic acid, acid halide or acid anhydride thereof;

wherein the modifier comprises a material having a structure composed of a first residue selected from the group consisting of divalent residues represented by Formula (I):

—Ar—W¹ₓ—Ar— (I)

where each Ar independently denotes a phenylene or naphthylene group, and may be independently substituted by at least one group and/or atom selected from the group consisting of a saturated or unsaturated acyclic or cyclic alkyl group having 1 to 12 carbon atoms, a saturated or unsaturated acyclic or cyclic alkoxy group having 1 to 12 carbon atoms, a halogen atom, a nitro group, an —SR¹ group (where R¹ denotes a hydrogen atom or an alkyl group having 1 to 4 carbon atoms), and an —R³—NR²₂ group (where each R² independently denotes a hydrogen atom or an alkyl group having 1 to 4 carbon atoms, and R³ denotes an alkylene group having 1 to 4 carbon atoms or may be absent), and x is 0 or 1, where when x is 1, W¹ denotes a saturated or unsaturated acyclic or cyclic alkylene group having 1 to 30 carbon atoms, an oxygen atom, a sulfur atom, sulfinyl group, or sulfonyl group, and when x is 0, two Ar's are covalently bonded by a carbon atom contained in each Ar;
and represented by Formula (II):

—Ar— (II)

where Ar denotes a phenylene group or naphthylene group, and Ar may be substituted by at least one group and/or atom selected from the group consisting of a saturated or unsaturated acyclic or cyclic alkyl group having 1 to 12 carbon atoms, a saturated or unsaturated acyclic or cyclic alkoxy group having 1 to 12 carbon atoms, a halogen atom, a nitro group, an —SR¹ group (where R¹ denotes a hydrogen atom or an alkyl group having 1 to 4 carbon atoms), a —R³—NR²₂ group (where each R² independently denotes a hydrogen atom or an alkyl group having 1 to 4 carbon atoms, and R³ denotes an alkylene group having 1 to 4 carbon atoms or may be absent), and a phenyl group optionally substituted by an alkyl group having 1 to 4 carbon atoms;
the first residue being bonded to two identical or different second residues selected from the group consisting of monovalent residues represented by Formula (III):

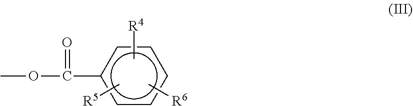

where R⁴, R⁵, and R⁶ each independently denotes a hydrogen atom or a saturated or unsaturated acyclic alkyl group having 1 to 5 carbon atoms;
and represented by Formula (IV):

—O—C(O)—R⁷— (IV)

where R⁷ denotes a saturated or unsaturated acyclic alkyl group having 1 to 5 carbon atoms.

As preferred aspects of the present invention, mention may be made of:

(2) The modifier for aromatic polyesters according to Aspect (1), wherein, in Formula (I), each Ar independently denotes a phenylene or naphthylene group, and may be independently substituted by at least one group and/or atom selected from the group consisting of a saturated or unsaturated acyclic or cyclic alkyl group having 1 to 6 carbon atoms, a saturated or unsaturated acyclic or cyclic alkoxy group having 1 to 6 carbon atoms, a halogen atom, a nitro group, an —SR¹ group (where R¹ denotes a hydrogen atom or an alkyl group having 1 to 4 carbon atoms), and an —R³—NR²₂ group (where each R² independently denotes a hydrogen atom or an alkyl group having 1 to 4 carbon atoms, and R³ denotes an alkylene group having 1 to 4 carbon atoms or may be absent), and x is 0 or 1, where when x is 1, W¹ denotes a saturated or unsaturated acyclic or cyclic alkylene group having 1 to 6 carbon atoms, an oxygen atom, a sulfur atom, a sulfinyl group, or a sulfonyl group, and when x is 0, two Ar's are covalently bonded by a carbon atom contained in each Ar;

(3) The modifier for aromatic polyesters according to Aspect (1), wherein, in Formula (I), each Ar independently denotes a phenylene or naphthylene group, and may be independently substituted by at least one group and/or atom selected from the group consisting of a saturated acyclic alkyl group having 1 to 4 carbon atoms, a saturated acyclic alkoxy group having 1 to 4 carbon atoms, a halogen atom, a nitro group, an —SR¹ group (where R¹ denotes a hydrogen atom or an alkyl group having 1 to 4 carbon atoms), and an —R³—NR²₂ group (where each R² independently denotes a hydrogen atom or an alkyl group having 1 to 4 carbon atoms, and R³ denotes an alkylene group having 1 to 4 carbon atoms or may be absent), and x is 0 or 1, where when x is 1, W¹ denotes a saturated acyclic alkylene group having 1 to 4 carbon atoms, an oxygen atom, a sulfur atom, a sulfinyl group, or a sulfonyl group, and when x is 0, two Ar's are covalently bonded by a carbon atom contained in each Ar;

(4) The modifier for aromatic polyesters according to Aspect (1), wherein, in Formula (I), each Ar independently denotes a phenylene or naphthylene group, and may be independently substituted by at least one group and/or atom selected from the group consisting of a saturated acyclic alkyl group having 1 to 4 carbon atoms, and a halogen atom, x is 1, and W¹ denotes a saturated acyclic alkylene group having 1 to 4 carbon atoms;

(5) The modifier for aromatic polyesters according to Aspect (1), wherein, in Formula (I), each Ar denotes a phenylene group, and may be independently substituted by a saturated acyclic alkyl group having 1 to 4 carbon atoms, x is 1, and W¹ denotes a saturated acyclic alkylene group having 1 to 4 carbon atoms;

(6) The modifier for aromatic polyesters according to Aspect (1), in Formula (I), each Ar denotes a phenylene group, x is 1, and W¹ denotes a saturated acyclic alkylene group having 1 to 3 carbon atoms;

(7) The modifier for aromatic polyesters according to any one of Aspects (1) to (6), wherein, in Formula (II), Ar denotes a phenylene group or a naphthylene group, and Ar may be substituted by at least one group and/or atom selected from the group consisting of a saturated or unsaturated acyclic or cyclic alkyl group having 1 to 6 carbon atoms, a saturated or unsaturated acyclic or cyclic alkoxy group having 1 to 6 carbon atoms, a halogen atom, a nitro group, an —SR¹ group (where R¹ denotes a hydrogen atom or an alkyl group having 1 to 4 carbon atoms), a —R³—NR²₂ group (where each R² independently denotes a hydrogen atom or an alkyl group having 1 to 4 carbon atoms, and R³ denotes an alkylene group having 1 to 4 carbon atoms or may be absent), and a phenyl group optionally substituted by an alkyl group having 1 to 4 carbon atoms;

(8) The modifier for aromatic polyesters according to any one of Aspects (1) to (6), wherein, in Formula (II), Ar denotes a phenylene group or a naphthylene group, and Ar may be substituted by at least one group and/or atom selected from the group consisting of a saturated acyclic alkyl group having 1 to 4 carbon atoms, a saturated acyclic alkoxy group having 1 to 4 carbon atoms, a halogen atom, a nitro group, an —SR¹ group (where R¹ denotes a hydrogen atom or an alkyl group having 1 to 4 carbon atoms), a —R³—NR²₂ group (where each R² independently denotes a hydrogen atom or an alkyl group having 1 to 4 carbon atoms, and R³ denotes an alkylene group having 1 to 4 carbon atoms or may be absent), and a phenyl group optionally substituted by an alkyl group having 1 to 4 carbon atoms;

(9) The modifier for aromatic polyesters according to any one of Aspects (1) to (6), wherein, in Formula (II), Ar denotes a phenylene group or a naphthylene group, and may be substituted by at least one group and/or atom selected from the group consisting of a saturated acyclic alkyl group having 1 to 4 carbon atoms and a halogen atom;

(10) The modifier for aromatic polyesters according to any one of Aspects (1) to (6), wherein, in Formula (II), Ar denotes a phenylene group, and may be substituted by a saturated acyclic alkyl group having 1 to 4 carbon atoms;

(11) The modifier for aromatic polyesters according to any one of Aspects (1) to (6), wherein, in Formula (II), Ar denotes a phenylene group;

(12) The modifier for aromatic polyesters according to any one of Aspects (1) to (11), wherein, in Formula (III), R⁴, R⁵, and R⁶ each independently denotes a hydrogen atom, or a saturated acyclic alkyl group having 1 to 5 carbon atoms;

(13) The modifier for aromatic polyesters according to any one of Aspects (1) to (11), wherein, in Formula (III), any one of R⁴, R⁵, and R⁶ denotes a hydrogen atom, and each of the other two groups independently denotes a saturated acyclic alkyl group having 1 to 5 carbon atoms;

(14) The modifier for aromatic polyesters according to any one of Aspects (1) to (13), wherein, in Formula (IV), R⁷ denotes a saturated acyclic alkyl group having 1 to 5 carbon atoms;

(15) The modifier for aromatic polyesters according to Aspect (1), being selected from the group consisting of diphenyl phthalate, diphenyl isophthalate, diphenyl terephthalate, bis(4-t-butylphenyl)phthalate, bis(4-t-butylphenyl)isophthalate, bis(4-t-butylphenyl)terephthalate, bis(6-t-butyl-2-methylphenyl)phthalate, bis(2-methyl-6-t-butylphenyl)isophthalate, bis(2-methyl-6-t-butylphenyl)terephthalate, 4,4′-isopropylidenediphenol di-4-t-butylbenzoate, 4,4′-isopropylidenediphenol di-2-t-butylbenzoate, and 4,4′-isopropylidenediphenol di-2,2′-dimethylpropionate;

(16) The modifier for aromatic polyesters according to Aspect (1), being selected from the group consisting of diphenyl phthalate, diphenyl isophthalate, diphenyl terephthalate, bis(4-t-butylphenyl)phthalate, bis(4-t-butylphenyl)isophthalate, bis(4-t-butylphenyl)terephthalate, bis(6-t-butyl-2-methylphenyl)phthalate, bis(2-methyl-6-t-butylphenyl)isophthalate, bis(2-methyl-6-t-butylphenyl)terephthalate, 4,4′-isopropylidenediphenol di-4-t-butylbenzoate, and 4,4′-isopropylidenediphenol di-2,2′-dimethylpropionate;

(17) The modifier for aromatic polyesters according to Aspect (1), being selected from the group consisting of bis(4-t-butylphenyl)phthalate, bis(4-t-butylphenyl)isophthalate, bis(4-t-butylphenyl)terephthalate, bis(6-t-butyl-2-methylphenyl)phthalate, bis(2-methyl-6-t-butylphenyl)isophthalate, bis(2-methyl-6-t-butylphenyl)terephthalate, 4,4′-isopropylidenediphenol di-4-t-butylbenzoate, and 4,4′-isopropylidenediphenol di-2,2′-dimethylpropionate;

(18) An aromatic polyester resin composition comprising the modifier for aromatic polyesters according to any one of Aspects (1) to (17);

(19) An aromatic polyester resin composition comprising the modifier for aromatic polyesters according to any one of Aspects (1) to (17) in an amount of 1 to 50 parts by weight based on 100 parts by weight of the aromatic polyesters;

(20) An aromatic polyester resin composition comprising the modifier for aromatic polyesters according to any one of Aspects (1) to (17) in an amount of 5 to 40 parts by weight based on 100 parts by weight of the aromatic polyesters;

(21) An aromatic polyester resin composition comprising the modifier for aromatic polyesters according to any one of Aspects (1) to (17) in an amount of 7 to 30 parts by weight based on 100 parts by weight of the aromatic polyesters;

(22) An aromatic polyester resin composition comprising the modifier for aromatic polyesters according to any one of Aspects (1) to (17) in an amount of 10 to 20 parts by weight based on 100 parts by weight of the aromatic polyesters;

(23) The aromatic polyester resin composition according to any one of Aspects (18) to (22), the aromatic polyester resin composition being for optical materials; and

(24) The aromatic polyester resin composition according to any one of Aspects (18) to (22), the aromatic polyester resin composition being for optical fibers;

The modifier for aromatic polyesters of the present invention can enhance the melt fluidity of aromatic polyesters without a significant decrease in the heat resistance of the aromatic polyesters. Accordingly, the aromatic polyester resin composition comprising the modifier for aromatic polyesters of the present invention can be molded at a lower temperature than that for conventional aromatic polyesters, resulting in effective prevention of coloration during molding processes.

### BRIEF DESCRIPTION OF THE DRAWINGS

FIG. 1 is an X-ray diffraction chart of aromatic polyester resin compositions of Examples 1 to 5, and of aromatic polyester resin of Comparative Example 1;

FIG. 2 is an X-ray diffraction chart of the aromatic polyester resin compositions containing varying amounts of a modifier for aromatic polyesters. In the drawings, reference numeral 1 represents an X-ray diffraction line of the aromatic polyester resin composition of Example 1, reference numeral 2 an X-ray diffraction line of the aromatic polyester resin composition of Example 2, numeral 3 an X-ray diffraction line of the aromatic polyester resin composition of Example 3, numeral 4 an X-ray diffraction line of the aromatic polyester resin composition of Example 4, numeral 5 an X-ray diffraction line of the aromatic polyester resin composition of Example 5, numeral 6 an X-ray diffraction line of the aromatic polyester resin composition of Comparative Example 1. Reference numeral 10 represents an X-ray diffraction line of the aromatic polyester resin composition containing 10 parts by weight of Modifier 1, reference numeral 20 an X-ray diffraction line of the aromatic polyester resin composition containing 20 parts by weight of Modifier 1, reference numeral 30 an X-ray diffraction line of the aromatic polyester resin composition containing 30 parts by weight of Modifier 1, reference numeral 40 an X-ray diffraction line of the aromatic polyester resin composition containing 40 parts by weight of Modifier 1, reference numeral 50 an X-ray diffraction line of the aromatic polyester resin composition containing 50 parts by weight of Modifier 1.

### DETAILED DESCRIPTION OF THE INVENTION

The modifier for aromatic polyesters of the present invention is compounded in aromatic polyesters comprising polyhydric phenol residues and residues of aromatic polycarboxylic acid, acid halide or acid anhydride thereof. The modifier for aromatic polyesters comprises a material having a structure composed of a first residue selected from the group consisting of divalent residues represented by Formula (I):

—Ar—W¹ₓ—Ar— (I)

and divalent residues represented by Formula (II):

—Ar— (II)

the first residue being bonded to two identical or different second residues selected from the group consisting of monovalent residues represented by Formula (III):

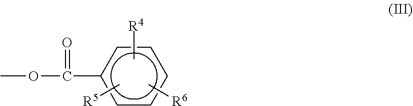

and monovalent residues represented by Formula (IV):

—O—C(O)—R⁷— (IV)

In Formula (I), each Ar independently denotes a phenylene or naphthylene group, and preferably a phenylene group. Each Ar may be independently substituted by at least one group and/or atom selected from the group consisting of a saturated or unsaturated acyclic or cyclic alkyl group having 1 to 12 carbon atoms, preferably a saturated or unsaturated acyclic or cyclic alkyl group having 1 to 6 carbon atoms, and more preferably a saturated acyclic alkyl group having 1 to 4 carbon atoms; a saturated or unsaturated acyclic or cyclic alkoxy group having 1 to 12 carbon atoms, preferably a saturated or unsaturated acyclic or cyclic alkoxy group having 1 to 6 carbon atoms, and more preferably a saturated acyclic alkoxy group having 1 to 4 carbon atoms; a halogen atom, preferably a fluorine, chlorine, bromine or iodine atom, and more preferably a chlorine atom; a nitro group; an —SR¹ group (where R¹ denotes a hydrogen atom or an alkyl group having 1 to 4 carbon atoms), preferably an —SH group; and an —R³—NR²₂ group (where each R² independently denotes a hydrogen atom or an alkyl group having 1 to 4 carbon atoms, and R³ denotes an alkylene group having 1 to 4 carbon atoms or may be absent), and preferably —NH₂. In Formula (I), x is 0 or 1, and preferably x is 1. When x is 1, W¹ denotes a saturated or unsaturated acyclic or cyclic alkylene group (including alkylidene group) having 1 to 30 carbon atoms, preferably a saturated or unsaturated acyclic or cyclic alkylene group (including alkylidene group) having 1 to 6 carbon atoms, more preferably a saturated acyclic alkylene group (including alkylidene group) having 1 to 4 carbon atoms, an oxygen atom, a sulfur atom, sulfinyl group, or sulfonyl group. When x is 0, two Ar's are covalently bonded by a carbon atom contained in each Ar. The divalent group represented by Formula (I) is not particularly limited as long as it is a divalent residue from which two hydroxyl groups of a divalent phenol used in the synthesis of aromatic polyesters are removed. For example, when x is 0, each Ar is a phenylene group, and when x is 1, each Ar is a phenylene group, and W¹ is an alkylene group such as a methylene, ethylene, propylene, or butylene group. Among these, preference is given to residues of 2,2-bis(4′-hydroxyphenyl)propane[bisphenol A], 1,1-bis(4′-hydroxyphenyl)ethane, and bis(4′-hydroxyphenyl)methane, and a residue of 2,2-bis(4′-hydroxyphenyl)propane[bisphenol A] is more preferable.

In Formula (II), Ar denotes a phenylene or naphthylene group, and preferably a phenylene group. The Ar may be substituted by at least one group and/or atom selected from the group consisting of a saturated or unsaturated acyclic or cyclic alkyl group having 1 to 12 carbon atoms, preferably a saturated or unsaturated acyclic or cyclic alkyl group having 1 to 6 carbon atoms, more preferably a saturated acyclic alkyl group having 1 to 4 carbon atoms; a saturated or unsaturated acyclic or cyclic alkoxy group having 1 to 12 carbon atoms, preferably a saturated or unsaturated acyclic or cyclic alkoxy group having 1 to 6 carbon atoms, more preferably a saturated acyclic alkoxy group having 1 to 4; a halogen atom, preferably a fluorine, chlorine, bromine or iodine atom, and more preferably a chlorine atom; a nitro group; an —SR¹ group (where R¹ denotes a hydrogen atom or an alkyl group having 1 to 4 carbon atoms), and preferably an —SH group; an —R³—NR²₂ group (where each R² independently denotes a hydrogen atom or an alkyl group having 1 to 4 carbon atoms, and R³ denotes an alkylene group having 1 to 4 carbon atoms or may be absent), preferably —NH₂; and a phenyl group optionally substituted by an alkyl group having 1 to 4 carbon atoms, and preferably a phenyl group. The divalent group represented by Formula (II) is not particularly limited as long as it is a divalent residue from which two hydroxyl groups of a divalent phenol used in the synthesis of aromatic polyesters are removed, or two —COOH groups of an aromatic dicarboxylic acid or its acid halide or acid anhydride used in the synthesis of aromatic polyesters are removed. Examples of Ar include a phenylene group and a naphthylene group, and preferably includes residues of hydroquinone, resorcinol, and catechol, and residues of phthaloyl dichloride, isophthaloyl dichloride and terephthaloyl dichloride.

In Formula (III), R⁴, R⁵, and R⁶ each independently denote a hydrogen atom or a saturated or unsaturated acyclic alkyl group having 1 to 5 carbon atoms, preferably R⁴, R⁵, and R⁶ each independently denote a hydrogen atom or a saturated acyclic alkyl group having 1 to 5 carbon atoms, and more preferably any one of R⁴, R⁵, and R⁶ denotes a hydrogen atom, and each of the other two groups independently denotes a saturated acyclic alkyl group having 1 to 5 carbon atoms; most preferably two of R⁴, R⁵, and R⁶ denote hydrogen atom, and the other group denotes a saturated acyclic alkyl group having 1 to 5 carbon atoms

In Formula (IV), R⁷ denotes a saturated or unsaturated acyclic alkyl group having 1 to 5 carbon atoms, and preferably a saturated acyclic alkyl group having 1 to 5 carbon atoms.

Any material may be used for the modifier for aromatic polyesters of the present invention as long as the material has a structure composed of a first residue selected from the group consisting of divalent residues represented by Formulae (I) and (II), the first residue being bonded to two second residues selected from the group consisting of monovalent residues represented by Formulae (III) and (IV). The two second residues selected from the group consisting of monovalent residues represented by Formulae (III) and (IV), to which the first residue selected from the group consisting of divalent residues represented by Formulae (I) and (II) is bonded, may be the same or different. As the modifier for aromatic polyesters, use is made of diphenyl phthalate, diphenyl isophthalate, diphenyl terephthalate, bis(4-t-butylphenyl)phthalate, bis(4-t-butylphenyl)isophthalate, bis(4-t-butylphenyl)terephthalate, bis(6-t-butyl-2-methylphenyl)phthalate, bis(2-methyl-6-t-butylphenyl)isophthalate, bis(2-methyl-6-t-butylphenyl)terephthalate, 4,4′-isopropylidenediphenol di-4-t-butylbenzoate, 4,4′-isopropylidenediphenol di-2-t-butylbenzoate, and 4,4′-isopropylidenediphenol di-2,2′-dimethylpropionate. Among these, use is preferably made of diphenyl phthalate, diphenyl isophthalate, diphenyl terephthalate, bis(4-t-butylphenyl)phthalate, bis(4-t-butylphenyl)isophthalate, bis(4-t-butylphenyl)terephthalate, bis(6-t-butyl-2-methylphenyl)phthalate, bis(2-methyl-6-t-butylphenyl)isophthalate, bis(2-methyl-6-t-butylphenyl)terephthalate, 4,4′-isopropylidenediphenol di-4-t-butylbenzoate, and 4,4′-isopropylidenediphenol di-2,2′-dimethylpropionate.

These modifiers for aromatic polyesters of the present invention can be compounded to any type of aromatic polyesters without limitation. Preferably, the modifier for aromatic polyesters has a similar structure as that contained in the aromatic polyester compounded therewith. Examples of the modifiers for the aromatic polyesters derived from the reaction of bisphenol A with isophthaloyl dichloride and terephthaloyl dichloride preferably include materials having a structure composed of a first residue selected from the group consisting of the residue represented by Formula (I) where each Ar is a phenylene group, and W¹ is a propylidene group, and the residue represented by Formula (II) where Ar is a phenylene group, the first residue being bonded to residues represented by Formula (III) and/or Formula (IV).

Aromatic polyesters comprising polyhydric phenol residues and residues of aromatic polycarboxylic acid, acid halide or acid anhydride thereof, which are compounded with the modifiers of the present invention, are known. The aromatic polyesters can be prepared, for example, by the reaction of polyhydric phenol with aromatic polycarboxylic acid, acid halide, or acid anhydride thereof. Polyhydric phenols, and aromatic polycarboxylic acid, or its acid halide or acid anhydride are known in the art. A preferable aromatic polyester is a terminal-protected polyester disclosed in Japanese Patent Application Laid-Open No. 2007-320989. The conditions for preparing aromatic polyesters are also known in the art. For example, the polyester can be prepared at a temperature of −10 to 55° C., for 0.01 to 24 hours under a pressure of 0.01 to 2 MPa, preferably in a nitrogen atmosphere. The preparation may be performed either batchwise or continuously.

As the polyhydric phenol, for example, use is made of 2,2′-dihydroxybiphenyl, 3,3′-dihydroxybiphenyl, 3,4′-dihydroxybiphenyl, 4,4′-dihydroxydiphenyl ether, 2,2-bis-(4′-hydroxyphenyl)propane[bisphenol A], 2,4′-dihydroxydiphenylmethane, bis-(4-hydroxyphenyl)methane, bis-(2-hydroxyphenyl)methane, 3,3′-methylidenebisphenol, 1,1-bis-(4′-hydroxyphenyl)ethane, 1,1-bis-(4′-hydroxyphenyl)cyclohexane, 1,2-bis-(4′-hydroxyphenyl)ethane, 1,1-bis-(4′-hydroxy-2′-chlorophenyl)ethane, 2,2-bis-(3′,5′-dimethyl-4′-hydroxyphenyl)propane, 2,2-bis-(4′-hydroxyphenyl)pentane, bis-(4′-hydroxyphenyl)ether, 4,3′-dihydroxydiphenyl ether, 4,2′-dihydroxydiphenyl ether, 2,2′-dihydroxydiphenyl ether, 2,3′-dihydroxydiphenyl ether, 4,4′-dihydroxy-2,6-dimethyl diphenyl ether, 3,4′-dihydroxydiphenyl, bis-(4-hydroxyphenyl)sulfone, bis-(3-hydroxyphenyl)sulfone, 2,4′-dihydroxydiphenylsulfone, 2,2′-methylidenebis(4-methylphenol), 2,2′-methylidenebis(5-methylphenol), 2,2′-methylidenebis(6-methylphenol), 4,4′-methylidenebis(2-methylphenol), 4,4′-methylidenebis(3-methylphenol), 2,2′-methylidenebis(4,6-dimethylphenol), 2,2′-methylidenebis(3,5-dimethylphenol), 4,4′-methylidenebis(2,6-dimethylphenol), 3,3′-methylidenebis(2,4,6-trimethylphenol), 2,2′-methylidenebis(4-propylphenol), 4,4′-methylidenebis(2-propylphenol), 4,4′-methylidenebis(2-methyl-6-ethylphenol), 2,2′-methylidenebis(3,4,5,6-tetramethylphenol), 4,4′-methylidenebis(2,3,5,6-tetramethylphenol), 2,2′-methylidenebis(4-tert-butylphenol), 4,4′-methylidenebis(2-methyl-5-isopropylphenol), 4,4′-methylidenebis(3-methyl-6-isopropylphenol), 4,4′-methylidenebis(5-methyl-6-isopropylphenol), 2,2′-methylidenebis(4-tert-butyl-6-methylphenol), 2,2′-methylidenebis(6-tert-butyl-4-methylphenol), 4,4′-methylidenebis(4-tert-butyl-6-methylphenol), 4,4′-methylidenebis(2-tert-butyl-5-methylphenol), 2,2′-methylidenebis(3,4-dimethyl-6-isopropylphenol), 2,2′-methylidenebis(6-tert-butyl-4-methylphenol), 2,2′-methylidenebis(4-(1,1,3,3-tetramethylbutyl)phenol), 2,2′-methylidenebis(4,6-ditert-butylphenol), 2,2′-methylidenebis(4,6-tert-butylphenol), 4,4′-methylidenebis(2,6-tert-butylphenol), 4,4′-methylidenebis(3,5-ditert-butylphenol), 2,2′-methylidenebis(4-chlorophenol), 4,4′-methylidenebis(2-chlorophenol), 2,2′-methylidenebis(4-bromophenol), 2,2′-methylidenebis(4,6-dichlorophenol), 2,2′-methylidenebis(4,5-dichlorophenol), 3,3′-methylidenebis(4,5-dichlorophenol), 4,4′-methylidenebis(2,5-dichlorophenol), 4,4′-methylidenebis(2,6-dichlorophenol), 2,2′-methylidenebis(4,6-dibromophenol), 4,4′-methylidenebis(2,6-dibromophenol), 2,2′-methylidenebis(3,4,6-trichlorophenol), 3,3′-methylidenebis(2,4,6-trichlorophenol), 2,2′-methylidenebis(6-bromo-4-chlorophenol), 2,2′-methylidenebis(4-bromo-6-nitrophenol), 2,2′-methylidenebis(6-chloro-4-nitrophenol), 2,2′-methylidenebis(4-nitrophenol), 4,4′-methylidenebis(2-nitrophenol), 2,2′-methylidenebis(4,6-dinitrophenol), 3,3′-methylidenebis(6-methoxyphenol), 4,4′-methylidenebis(2-methoxyphenol), bis-(4-hydroxy-2,6-dimethyl-3-methoxyphenyl)methane, 2,2′-methylidenebis(4-chloro-6-methylphenol), 2,2′-methylidenebis(6-chloro-4-methylphenol), 4,4′-methylidenebis(2-chloro-6-methylphenol), 2,2′-methylidenebis(6-bromo-4-methylphenol), 4,4′-methylidenebis(6-bromo-2-methylphenol), 2,2′-methylidenebis(4-chloro-3,5-dimethylphenol), 2,2′-methylidenebis(3-chloro-4,6-dimethylphenol), 2,2′-methylidenebis(6-bromo-4,5-dimethylphenol), 4,4′-methylidenebis(2-chloro-3,5,6-trimethylphenol), 2,2′-methylidenebis(4-chloro-6-isopropylphenol), 2,2′-methylidenebis(6-chloro-4-tert-butylphenol), 2,2′-methylidenebis(4-chloro-3-methyl-6-isopropylphenol), 2,2′-methylidenebis(4-chloro-6-tert-butyl-3-methylphenol), 2,2′-methylidenebis(4,6-dichloro-3-methylphenol), 2,2′-methylidenebis(6-nitro-4-tert-butylphenol), 2,2′-isopropylidenebis(5-methylphenol), 4,4′-isopropylidenebis(2-methylphenol), 4,4′-isopropylidenebis(2-cyclohexylphenol), 4,4′-isopropylidenebis(2,6-dibromophenol), 4,4′-isopropylidenebis(2-nitrophenol), 4,4′-isopropylidenebis(2,6-dinitrophenol), 4,4′-butanediylbis(2-methylphenol), 4,4′-butylidenebisphenol, 2,2′-butylidenebis(6-tert-butyl-4-methylphenol), 4,4′-butylidenebis(6-tert-butyl-2-methylphenol), 4,4′-sec-butylidenebisphenol, 4,4′-sec-butylidenebis(3-methylphenol), 2,2′-sec-butylidenebis(3-methyl-6-isopropylphenol), 2,2′-sec-butylidenebis(6-tert-butyl-4-methylphenol), 4,4′-isobutylidenebisphenol, 4,4′-isobutylidenebis(6-tert-butyl-4-methylphenol), 4,4′-(1,3-cyclohexanediyl)bisphenol, 4,4′-cyclohexylidenebisphenol, 4,4′-cyclohexylidenebis(2-chlorophenol), 4,4′-cyclohexylidenebis(2,6-dichlorophenol), 2,2′-thiobisphenol, 4,4′-thiobisphenol, 4,4′-thiobis(2-methylphenol), 2,2′-thiobis(4,5-dimethylphenol), 2,2′-thiobis(4,6-dimethylphenol), 4,4′-thiobis(2,6-dimethylphenol), 2,2′-thiobis(6-tert-butyl-4-methylphenol), 4,4′-thiobis(2-tert-butyl-5-methylphenol), 2,2′-thiobis(4-fluorophenol), 2,2′-thiobis(4-chlorophenol), 4,4′-thiobis(3-chlorophenol), 2,2′-thiobis(4-chloro-5-methylphenol), 2,2′-thiobis(4,6-dichlorophenol), 4,4′-thiobis(2-bromophenol), 2,2′-thiobis(5-nitrophenol), 4,4′-sulfinylbisphenol, 4,4′-sulfinylbis(2-methylphenol), 4,4′-sulfinylbis(2-tert-butyl-5-methylphenol), 4,4′-sulfinylbis(2-chlorophenol), 4,4′-sulfinylbis(4-chlorophenol), 2,2′-sulfinylbis(4,6-dichlorophenol), 4,4′-sulfinylbis(2-bromophenol), 2,2′-sulfonylbisphenol, 4,4′-sulfonylbisphenol, 4,4′-sulfonylbis(2-methylphenol), 4,4′-sulfonylbis(2,5-dimethylphenol), 4,4′-sulfonylbis(2-tert-butyl-5-methylphenol), 4,4′-sulfonylbis(2-chlorophenol), 4,4′-sulfonylbis(3-chlorophenol), 4,4′-sulfonylbis(2-bromophenol), 4,4′-sulfonylbis(2-nitrophenol), 1,1′-binaphthalene-2,2′-diol, 2,2-bis-(4-hydroxynaphthyl)propane, bis-(hydroxynaphthyl)ether, and 2,2′-ethanediyldimercaptobisphenol. In addition, as the other polyhydric phenol, for example, use is made of 1,2-dihydroxynaphthalene, 1,3-dihydroxynaphthalene, 1,4-dihydroxynaphthalene, 1,5-dihydroxynaphthalene, 1,6-dihydroxynaphthalene, 1,7-dihydroxynaphthalene, 1,8-dihydroxynaphthalene, 2,3-dihydroxynaphthalene, 2,6-dihydroxynaphthalene, 2,7-dihydroxynaphthalene, catechol, 3-methylcatechol, 4-methylcatechol, 3-ethylcatechol, 4-ethylcatechol, 3-n-propylcatechol, 4-n-propylcatechol, 3-(t-butyl)catechol, 3-n-pentylcatechol, 4-n-pentylcatechol, 4-(1,1-dimethylpropyl)catechol, 4-hexylcatechol, 4-cyclohexylcatechol, 4-(1,1,3,3-tetramethylbutyl)catechol, 4-nonylcatechol, 3,4-dimethylcatechol, 3,5-dimethylcatechol, 3,6-dimethylcatechol, 4,5-dimethylcatechol, 4-methyl-5-ethylcatechol, 3-chlorocatechol, 4-chlorocatechol, 3-bromocatechol, 4-bromocatechol, 3-fluorocatechol, 4-fluorocatechol, 3,5-dichlorocatechol, 4,5-dichlorocatechol, 3,4-dichlorocatechol, 3,4-dibromocatechol, 3,5-dibromocatechol, 4,5-dibromocatechol, 4-chloro-5-nitrocatechol, 3-chloro-6-methoxycatechol, 5-bromo-4-nitrocatechol, 4-bromo-5-methylcatechol, 3-bromo-5-(t-butyl)catechol, 3,4,5-trichlorocatechol, 3,4,5-tribromocatechol, 3,4,6-tribromocatechol, tetrachlorocatechol, tetrabromocatechol, 3-aminocatechol, 4-aminocatechol, 3-(2-aminoethyl)catechol, 4-(2-methylaminoethyl)catechol, 4-(2-dimethylaminoethyl)catechol, 4-(2-aminoethyl)catechol, 6-amino-4-(2-aminoethyl)catechol, 3-nitrocatechol, 3,4-dinitrocatechol, 4,5-dinitrocatechol, 3-nitro-6-methoxycatechol, 4-nitro-3-methoxycatechol, 5-nitro-3-methylcatechol, 4-methoxycatechol, 6-methoxycatechol, 3-propioxycatechol, 3-butyloxycatechol, 3,4-dimethoxycatechol, 3,6-dimethoxycatechol, 5-methoxy-3-(t-butyl)catechol, 3-ethoxy-(t-butyl)catechol, 3,4,6-trimethoxycatechol, resorcinol, 2-chlororesorcinol, 4-chlororesorcinol, 5-chlororesorcinol, 2,4-dichlororesorcinol, 4,6-dichlororesorcinol, 2,4,6-trichlororesorcinol, 2-bromo-4-chlororesorcinol, 4-bromo-2-chlororesorcinol, 4-chloro-5-methylresorcinol, 6-chloro-4-ethylresorcinol, 2-chloro-4-butylresorcinol, 6-chloro-4-butylresorcinol, 6-chloro-4-cyclohexylresorcinol, 2,4-dichloro-5-methylresorcinol, trichlororesorcinol, 2-bromoresorcinol, 4-bromoresorcinol, 5-bromoresorcinol, 2,4-dibromoresorcinol, 4,6-dibromoresorcinol, 2,4,6-tribromoresorcinol, 6-bromo-4-butylresorcinol, 2-iodoresorcinol, 4-iodoresorcinol, 5-iodoresorcinol, 4,6-diiodoresorcinol, 2,4,6-triiodoresorcinol, 2-aminoresorcinol, 5-aminoresorcinol, 4-amino-2,5-dimethylresorcinol, 5-mercaptoresorcinol, 5-methylthioresorcinol, 5-ethylthioresorcinol, 5-propylthioresorcinol, 5-butylthioresorcinol, 2-nitroresorcinol, 4-nitroresorcinol, 5-nitroresorcinol, 2,4-dinitroresorcinol, 4,6-dinitroresorcinol, 2,4,6-trinitroresorcinol, tetranitroresorcinol, 6-nitro-5-methoxyresorcinol, 2-nitro-5-methoxyresorcinol, 4-nitro-5-methoxyresorcinol, 2,4-dinitro-5-methylresorcinol, 2,4,6-trinitro-5-methylresorcinol, 2-methoxyresorcinol, 4-methoxyresorcinol, 5-methoxyresorcinol, 2,3-methoxyresorcinol, 2,5-methoxyresorcinol, 2-methoxy-5-methylresorcinol, 5-methoxy-4-methylresorcinol, 5-methoxy-6-methylresorcinol, 5-ethoxyresorcinol, 2-methylresorcinol, 4-methylresorcinol, 5-methylresorcinol, 2-ethylresorcinol, 4-ethylresorcinol, 5-ethylresorcinol, 2-n-propylresorcinol, 4-n-propylresorcinol, 5-n-propylresorcinol, 2-(2-propenyl)resorcinol, 4-(2-propenyl)resorcinol, 4-(1-methylethenyl)resorcinol, 4-(2-methylethenyl)resorcinol, 2-n-butylresorcinol, 4-n-butylresorcinol, 5-n-butylresorcinol, 2-n-butylresorcinol, 5-sec-butylresorcinol, 4-tert-butylresorcinol, 2-n-pentylresorcinol, 4-n-pentylresorcinol, 5-n-pentylresorcinol, 4-(1-methylbutyl)resorcinol, 5-(2-methyl-1-ethylpropyl)resorcinol, 2-n-hexylresorcinol, 4-n-hexylresorcinol, 5-n-hexylresorcinol, 4-(4-methylpentyl)resorcinol, 5-(4-methylpentyl)resorcinol, 5-(1,1-dimethylbutyl)resorcinol, 5-(1,2-dimethylbutyl)resorcinol, 5-(1-methyl-1-pentenyl)resorcinol, 4-cyclohexylresorcinol, 4-phenylresorcinol, 4-heptylresorcinol, 5-heptylresorcinol, 5-(1-methylhexyl)resorcinol, 4-phenylmethylresorcinol, 2-octylresorcinol, 4-octylresorcinol, 5-octylresorcinol, 4-(1-methylheptyl)resorcinol, 4-(1,1,3,3-tetramethylbutyl)resorcinol, 4-(2-phenylethyl)resorcinol, 5-nonylresorcinol, 5-(1-methyloctyl)resorcinol, 5-(1,1-dimethylheptyl)resorcinol, 5-(1,2-dimethylheptyl)resorcinol, 5-(1,2,4-trimethylhexyl)resorcinol, 4-decylresorcinol, 5-(1-methylnonyl)resorcinol, 2,4-dimethylresorcinol, 2,5-dimethylresorcinol, 4,5-dimethylresorcinol, 4,6-dimethylresorcinol, 4-ethyl-2-methylresorcinol, 5-ethyl-2-methylresorcinol, 2-ethyl-4-methylresorcinol, 5-ethyl-4-methylresorcinol, 6-ethyl-4-methylresorcinol, 5-ethenyl-4-methylresorcinol, 2,4-dimethylresorcinol, 5-methyl-4-propylresorcinol, 2-methyl-5-sec-butylresorcinol, 4,6-di(isopropyl)resorcinol, 4-ethyl-6-pentylresorcinol, 4,6-di(tert-butyl)resorcinol, 2,4,5-trimethylresorcinol, 2,4,6-trimethylresorcinol, 4,5,6-trimethylresorcinol, 4,6-dimethyl-5-sec-butylresorcinol, tetramethylresorcinol, 5-trifluoromethylresorcinol, hydroquinone, phenylhydroquinone, chlorohydroquinone, methylhydroquinone, trifluorohydroquinone, tetrafluorohydroquinone, 2-chloro-3-methoxyhydroquinone, 2-chloro-5-methoxyhydroquinone, 2-chloro-6-methoxyhydroquinone, 2,3-dichlorohydroquinone, 2,5-dichlorohydroquinone, 2,6-dichlorohydroquinone, trichlorohydroquinone, tetrachlorohydroquinone, bromohydroquinone, 3-bromo-2,6-dimethylhydroquinone, 2,5-dibromohydroquinone, 2,6-dibromohydroquinone, tribromohydroquinone, iodohydroquinone, 2,6-diiodohydroquinone, tetraiodohydroquinone, nitrohydroquinone, 2,6-dinitrohydroquinone, methoxyhydroquinone, 2-methoxy-3-methylhydroquinone, 2-methoxy-5-methylhydroquinone, 3-methoxy-2-methylhydroquinone, 5-methoxy-2-methylhydroquinone, 2-methoxy-6-propylhydroquinone, 2-methoxy-5-propenylhydroquinone, 2,3-dimethoxyhydroquinone, 2,5-dimethoxyhydroquinone, 2,6-dimethoxyhydroquinone, mercaptohydroquinone, methylhydroquinone, 2-methyl-6-ethylhydroquinone, 2-methyl-5-isopropylhydroquinone, 2-methyl-5-cyclohexylhydroquinone, 2,3-dimethylhydroquinone, 2,5-dimethylhydroquinone, 2,6-dimethylhydroquinone, trimethylhydroquinone, tetramethylhydroquinone, ethylhydroquinone, 2,6-diethylhydroquinone, vinylhydroquinone, n-propylhydroquinone, isopropylhydroquinone, 1-propenylhydroquinone, 2-propenylhydroquinone, 2,5-diisopropylhydroquinone, 4-butylhydroquinone, 2,3-di(tert-butyl)hydroquinone, 2,5-di(tert-butyl)hydroquinone, 2,6-di(tert-butyl)hydroquinone, hexylhydroquinone, 4-methylpentylhydroquinone, cyclohexylhydroquinone, 4-phenylmethylhydroquinone, and octylhydroquinone. Among these, use are preferably made of 2,2-bis-(4′-hydroxyphenyl)propane[bisphenol A], 1,1-bis-(4′-hydroxyphenyl)ethane, bis-(4-hydroxyphenyl)methane, hydroquinone, resorcinol, catechol. More preferably, use is made of 2,2-bis-(4′-hydroxyphenyl)propane[bisphenol A].

The aromatic polycarboxylic acid, or an acid halide or acid anhydride thereof is mentioned below. As an aromatic dicarboxylic acid, for example, use is made of phthalic acid, dimethyl phthalate, diphenyl phthalate, isophthalic acid, dimethyl isophthalate, di(cyanomethyl)isophthalate, diphenyl isophthalate, di(2,4-dinitrophenyl)isophthalate, (1,1-dioxobenzothiophene-3-yl)isophthalate, di(3-benzoisoxazolyl)isophthalate, di(2-benzothiazolyl)isophthalate, (1-benzotriazolyl)isophthalate, S,S′-dipropyl dithioisophthalate, S,S′-di(p-nitrophenyl)dithioisophthalate, S,S′-di(2-benzoxazolyl)dithioisophthalate, S,S′-di(2-benzothiazolyl)dithioisophthalate, 4-methylisophthalic acid, dimethyl isophthalate, 5-methylisophthalic acid, dimethyl 5-methylisophthalate, 4,5-dimethylisophthalic acid, 4,6-dimethylisophthalic acid, 4-chloroisophthalic acid, dimethyl 4-chloroisophthalate, 5-chloroisophthalic acid, dimethyl 5-chloroisophthalate, 4,6-dichloroisophthalic acid, dimethyl 4,6-dichloroisophthalate, 4-bromoisophthalic acid, 4,6-dibromoisophthalic acid, dimethyl 4,6-dibromoisophthalate, terephthalic acid, dimethyl terephthalate, di(cyanomethyl)terephthalate, diphenyl terephthalate, di(3-benzoisoxazolyl)terephthalate, di(2-benzothiazolyl)terephthalate, 2-methylterephthalic acid, dimethyl 2-methylterephthalate, 2,5-dimethylterephthalic acid, 2,6-dimethylterephthalic acid, dimethyl 2,6-dimethylterephthalate, 2-chloroterephthalic acid, dimethyl 2-chloroterephthalate, 2,5-dichloroterephthalic acid, dimethyl 2,5-dichloroterephthalate, tetrachloroterephthalic acid, dimethyl tetrachloroterephthalate, 2-bromoterephthalic acid, dimethyl 2-bromoterephthalate, 2,5-dibromoterephthalic acid, diethyl 2,5-dibromoterephthalate, 2,2′-diphenyldicarboxylic acid, 3,3′-diphenyldicarboxylic acid, 3,4′-diphenyldicarboxylic acid, 4,4′-diphenyldicarboxylic acid, 3,4′-dicarboxydiphenyl ether, 4,4′-dicarboxydiphenyl ether, 1,2-naphthalenedicarboxylic acid, dimethyl 1,2-naphthalenedicarboxylate, 1,3-naphthalenedicarboxylic acid, 1,4-naphthalenedicarboxylic acid, dimethyl 1,4-naphthalenedicarboxylate, 1,5-naphthalenedicarboxylic acid, dimethyl 1,5-naphthalene dicarboxylate, 1,6-naphthalene dicarboxylic acid, dimethyl 1,6-naphthalenedicarboxylate, diphenyl 1,6-naphthalenedicarboxylate, 1,7-naphthalenedicarboxylic acid, dimethyl 1,7-naphthalenedicarboxylate, 1,8-naphthalenedicarboxylic acid, dimethyl 1,8-naphthalenedicarboxylate, diphenyl 1,8-naphthalenedicarboxylate, 2,3-naphthalenedicarboxylic acid, dimethyl 2,3-naphthalenedicarboxylate, diphenyl 2,3-naphthalenedicarboxylate, 2,6-naphthalenedicarboxylic acid, dimethyl 2,6-naphthalenedicarboxylate, diphenyl 2,6-naphthalenedicarboxylate, 2,7-naphthalenedicarboxylic acid, diphenyl 2,7-naphthalenedicarboxylate, 1,2-(9-oxofluorene)dicarboxylic acid, dimethyl 1,2-(9-oxofluorene)dicarboxylate, dimethyl 1,5-(9-oxofluorene)dicarboxylate, 1,6-(9-oxofluorene)dicarboxylic acid, dimethyl 1,6-(9-oxofluorene)dicarboxylate, 1,7-(9-oxofluorene)dicarboxylic acid, dimethyl 1,7-(9-oxofluorene)dicarboxylate, 2,3-(9-oxofluorene)dicarboxylic acid, dimethyl 2,3-(9-oxofluorene)dicarboxylate, 2,7-(9-oxofluorene)dicarboxylic acid, dimethyl 2,7-(9-oxofluorene)dicarboxylate, 1,4-anthracenedicarboxylic acid, 1,5-anthracenedicarboxylic acid, diethyl 1,5-anthracenedicarboxylate, 1,8-anthracenedicarboxylic acid, 1,9-anthracenedicarboxylic acid, 2,3-anthracenedicarboxylic acid, 9,10-anthracenedicarboxylic acid, dimethyl 9,10-anthracenedicarboxylate, 1,2-anthraquinonedicarboxylic acid, dimethyl 1,2-anthraquinonedicarboxylate, 1,3-1,2-anthraquinonedicarboxylic acid, 1,4-anthraquinonedicarboxylic acid, 1,5-anthraquinonedicarboxylic acid, dimethyl 1,5-anthraquinonedicarboxylate, diphenyl 1,5-anthraquinonedicarboxylate, 1,6-anthraquinonedicarboxylic acid, 1,7-anthraquinonedicarboxylic acid, 1,8-anthraquinonedicarboxylic acid, 2,3-anthraquinonedicarboxylic acid, 2,7-anthraquinonedicarboxylic acid, 2,3-biphenyldicarboxylic acid, dimethyl 2,3-biphenyldicarboxylate, 2,5-biphenyldicarboxylic acid, 2,6-biphenyldicarboxylic acid, 3,4-biphenyldicarboxylic acid, dimethyl 3,4-biphenyldicarboxylate, 3,4-biphenyldicarboxylic acid, dimethyl 3,4-biphenyldicarboxylate, 3,4-biphenyldicarboxylic acid, 2,2′-biphenyldicarboxylic acid, dimethyl 2,2′-biphenyldicarboxylate, diphenyl 2,2′-biphenyldicarboxylate, 2,4′-biphenyldicarboxylic acid, dimethyl 2,4′-biphenyldicarboxylate, 3,3′-biphenyldicarboxylic acid, dimethyl 3,3′-biphenyldicarboxylate, 3,4′-biphenyldicarboxylic acid, dimethyl 3,4′-biphenyldicarboxylate, 4,4′-biphenyldicarboxylic acid, dimethyl 4,4′-biphenyldicarboxylate, diphenyl 4,4′-biphenyldicarboxylate, 1,5-biphenylenedicarboxylic acid, dimethyl 1,5-biphenylenedicarboxylate, 1,8-biphenylenedicarboxylic acid, dimethyl 1,8-biphenylenedicarboxylate, 2,6-biphenylenedicarboxylic acid, dimethyl 2,6-biphenylenedicarboxylate, 2,7-biphenylenedicarboxylic acid, dimethyl 2,7-biphenylenedicarboxylate, 2,2′-dimethyl-4,4′-biphenyldicarboxylic acid, diethyl 2,2′-dimethyl-4,4′-biphenyldicarboxylate, 4,4″-p-terphenyldicarboxylic acid, dimethyl 4,4″′-p-quaterphenyldicarboxylate, 4,4″′-p-quaterphenyldicarboxylic acid, 2,2′-methylenedibenzoic acid, dimethyl 2,2′-methylenedibenzoate, 2,4′-methylenedibenzoic acid, dimethyl 2,4′-methylenedibenzoate, 3,3′-methylenedibenzoic acid, 4,4′-methylenedibenzoic acid, dimethyl 4,4′-methylenedibenzoate, 4,4′-isopropylidenedibenzoic acid, 2,2′-bibenzyldicarboxylic acid, dimethyl 2,2′-benzyldicarboxylate, dimethyl 3,3′-bibenzyldicarboxylate, 4,4′-bibenzyldicarboxylic acid, dimethyl 4,4′-bibenzyldicarboxylate, 2,2′-trans-stilbenedicarboxylic acid, dimethyl 2,2′-trans-stilbenedicarboxylate, diphenyl 2,2′-trans-stilbenedicarboxylate, 2,4′-trans-stilbenedicarboxylic acid, 4,4′-trans-stilbenedicarboxylic acid, 4,4′-trans-stilbenedicarboxylic acid, 2,4′-trans-stilbenedicarboxylic acid, 4,4′-trans-stilbenedicarboxylic acid, dimethyl 4,4′-trans-stilbenedicarboxylate, 2,2′-tolanedicarboxylic acid, dimethyl 2,2′-tolanedicarboxylate, 2,4′-tolanedicarboxylic acid, 4,4′-tolanedicarboxylic acid, dimethyl 4,4′-tolanedicarboxylate, pseudo-p-dicarboxy[2,2]paracyclophane, 4,4′-carbonyldibenzoic acid, 3,3′-oxydibenzoic acid, 4,4′-oxydibenzoic acid, dimethyl 4,4′-oxydibenzoate, diphenyl 4,4′-oxydibenzoate, 4,4′-thiodibenzoic acid, 4,4′-sulfonyldibenzoic acid, dimethyl 4,4′-sulfonyldibenzoate, 3,3′-dithiodibenzoic acid, 4,4′-dithiodibenzoic acid, diethyl 4,4′-dithiodibenzoate, 2,2′-3,3′-dithiodibenzoic acid, 2,2′-azobenzenedicarboxylic acid, dimethyl 2,2′-azobenzenedicarboxylate, 3,3′-azobenzenedicarboxylic acid, dimethyl 3,3′-azobenzenedicarboxylate, 4,4′-azobenzenedicarboxylic acid, dimethyl 4,4′-azobenzenedicarboxylate, homophthalic acid, dimethyl homophthalate, homoisophthalic acid, dimethyl homoisophthalate, homoterephthalic acid, dimethyl homoterephthalate, o-phenylenediacetic acid, diethyl o-phenylenediacetate, m-phenylenediacetic acid, diethyl m-phenylenediacetate, p-phenylenediacetic acid, diethyl p-phenylenediacetate, 3,3′-o-phenylenedipropionic acid, diethyl 3,3′-o-phenylenedipropionate, 3,3′-m-phenylenedipropionic acid, diethyl 3,3′-m-phenylenediproionate, 3,3′-p-phenylenedipropionic acid, diethyl 3,3′-p-phenylenedipropionate, 2-carboxycinnamic acid, 3-carboxycinnamic acid, 4-carboxycinnamic acid, diethyl 4-carboxycinnamate, 3t,3′ t-o-phenylenediacrylic acid, dimethyl 3t,3′ t-o-phenylenediacrylate, 3t,3′ t-m-phenylenediacrylic acid, dimethyl 3t,3′ t-m-phenylenediacrylate, 3t,3′t-p-phenylenediacrylic acid, dimethyl 3t,3′ t-p-phenylenediacrylate, m-phenylenepropiolic acid, dimethyl m-phenylenepropiolate, 1,4-naphthalenediacetic acid, 1,5-naphthalenediacetic acid, dimethyl 1,5-naphthalenediacetate, 3,3′-(1,4-naphthalene)dipropionic acid, diethyl 3,3′-(1,4-naphthalene)dipropionate, 4,4′-biphenyldiacetic acid, diethyl 4,4′-biphenyldiacetate, 3,3′-(4,4′-biphenyl)dipropionic acid, 3,3′-[4,4′-(methylenedi-p-phenylene)]dipropionic acid, 4,4′-bibenzyldibutyric acid, 3,3′-(4,4′-bibenzyl)dipropionic acid, 4,4′-(oxydi-p-phenylene)dibutyric acid, 3,3′-[4,4′-(oxydi-p-phenylene)]dipropionic acid, 3,3′-[4,4′-(oxydi-p-phenylene)]dibutyric acid, diphenylsulfonedicarboxylic acid, 1,4-cyclohexanedicarboxylic acid, 2,3-furandicarboxylic acid, dimethyl 2,3-furandicarboxylate, 2,4-furandicarboxylic acid, dimethyl 2,4-furandicarboxylate, 2,5-furandicarboxylic acid, dimethyl 2,5-furandicarboxylate, diphenyl 2,5-furandicarboxylate, 3,4-furandicarboxylic acid, dimethyl 3,4-furandicarboxylate, 3,4-diphenyl-2,5-furandicarboxylic acid, dimethyl 3,4-diphenyl-2,5-furandicarboxylate, 3,3′-(2,5-furan)dipropionic acid, dimethyl 3,3′-(2,5-furan)dipropionate, 2,5-cis-tetrahydrofurandicarboxylic acid, dimethyl 2,5-cis-tetrahydrofurandicarboxylate, 3,3′-(2,5-cis-tetrahydrofuran)dipropionic acid, diethyl 3,3′-(2,5-cis-tetrahydrofuran)dipropionate, 2,3-thiophenedicarboxylic acid, dimethyl 2,3-thiophenedicarboxylate, 2,4-thiophenedicarboxylic acid, dimethyl 2,4-thiophenedicarboxylate, 2,5-thiophenedicarboxylic acid, dimethyl 2,5-thiophenedicarboxylate, diphenyl 2,5-thiophenedicarboxylate, 3,4-thiophenedicarboxylic acid, dimethyl 3,4-thiophenedicarboxylate, 3,4-diphenyl-2,5-thiophenedicarboxylic acid, dimethyl 3,4-diphenyl-2,5-thiophenedicarboxylate, 2,5-thiophenediacetic acid, 3,3′-(2,5-thiophene)dipropionic acid, diethyl 3,3′-(2,5-thiophene)dipropionate, 2,5-cis-tetrahydrothiophenedicarboxylic acid, diethyl 2,5-cis-tetrahydrothiophenedicarboxylate, 3,4-cis-tetrahydrothiophenedicarboxylic acid, dimethyl 3,4-cis-tetrahydrothiophenedicarboxylate, 1,1-dioxo-2,5-cis-tetrahydrothiophenedicarboxylic acid, diethyl 1,1-dioxo-2,5-cis-tetrahydrothiophenedicarboxylate, 2, 6-4H-pyranedicarboxylic acid, 4-oxo-2, 6-4H-pyranedicarboxylic acid, diethyl 4-oxo-2, 6-4H-pyranedicarboxylate, 2,6-cis-tetrahydropyranedicarboxylic acid, dimethyl 2,6-cis-tetrahydropyranedicarboxylate, 2,6-cis-tetrahydrothiopyranedicarboxylic acid, dimethyl 2,6-cis-tetrahydrothiopyranedicarboxylate, 1,1-dioxo-2,6-cis-tetrahydrothiopyranedicarboxylic acid, dimethyl 1,1-dioxo-2,6-cis-tetrahydrothiopyranedicarboxylate, 2,8-dibenzofurandicarboxylic acid, dimethyl 2,8-dibenzofurandicarboxylate, 3,7-dibenzofurandicarboxylic acid, dimethyl 3,7-dibenzofurandicarboxylate, 4,6-dibenzofurandicarboxylic acid, dimethyl 4,6-dibenzofurandicarboxylate, 2,8-dibenzothiophenedicarboxylic acid, 5,5-dioxo-2,8-dibenzothiophenedicarboxylic acid, 9-oxo-1,8-xanthenedicarboxylic acid, 9-oxo-2,7-xanthenedicarboxylic acid, dimethyl 9-oxo-2,7-xanthenedicarboxylate, 1,6-dibenzo[1,4]dioxindicarboxylic acid, dimethyl 1,6-dibenzo[1,4]dioxindicarboxylate, 2,7-dibenzo[1,4]dioxindicarboxylic acid, dimethyl 2,7-dibenzo[1,4]dioxindicarboxylate, 2,8-dibenzo[1,4]dioxindicarboxylic acid, dimethyl 2,8-dibenzo[1,4]dioxindicarboxylate, 1,6-phenoxathiindicarboxylic acid, 4,6-phenoxathiindicarboxylic acid, dimethyl 4,6-phenoxathiindicarboxylate, 10,10-dioxo-1,6-phenoxainedicarboxylic acid, dimethyl 10,10-dioxo-1,6-phenoxainedicarboxylate, 10,10-dioxo-1,9-phenoxainedicarboxylic acid, dimethyl 10,10-dioxo-1,9-phenoxainedicarboxylate, 10,10-dioxo-2,8-phenoxainedicarboxylic acid, dimethyl 10,10-dioxo-2,8-phenoxainedicarboxylate, 10,10-dioxo-4,6-phenoxainedicarboxylic acid, 2,7-thianthrenedicarboxylic acid, dimethyl 2,7-thianthrenedicarboxylate, 10,10-dioxo-1,9-thianthrenedicarboxylic acid, 5,5,10,10-tetraoxo-2,7-thianthrenedicarboxylic acid, dimethyl 5,5,10,10-tetraoxo-2,7-thianthrenedicarboxylate, 10-oxo-10-phenyl-2,8-phenoxaphosphinedicarboxylic acid, dimethyl 9-oxabicyclo[3,3,1]nonane-2,6-dicarboxylate, diphenyl 9-oxabicyclo[3,3,1]nonane-2,6-dicarboxylate, 2,4,6,8-tetraoxaspiro[5,5]undecane-3,9-dicarboxylic acid, dimethyl 2,4,6,8-tetraoxaspiro[5,5]undecane-3,9-dicarboxylate, 2,4,6,8-tetraoxaspiro[5,5]undecane-3,9-diacetic acid, diethyl 2,4,6,8-tetraoxaspiro[5,5]undecane-3,9-diacetate, 2,3-pyrroledicarboxylic acid, dimethyl 2,3-pyrroledicarboxylate, 2,4-pyrroledicarboxylic acid, dimethyl 2,4-pyrroledicarboxylate, 2,5-pyrroledicarboxylic acid, dimethyl 2,5-pyrroledicarboxylate, 1-methyl-2,5-pyrroledicarboxylic acid, dimethyl 1-methyl-2,5-pyrroledicarboxylate, 1-phenyl-2,5-pyrroledicarboxylic acid, dimethyl 1-phenyl-2,5-pyrroledicarboxylate, 3,4-pyrroledicarboxylic acid, dimethyl 3,4-pyrroledicarboxylate, 1-methyl-3,4-pyrroledicarboxylic acid, diethyl 1-methyl-3,4-pyrroledicarboxylate, 1-phenyl-3,4-pyrroledicarboxylic acid, diethyl 1-phenyl-3,4-pyrroledicarboxylate, diethyl 3,5-dimethyl-2,4-pyrroledicarboxylate, 2,5-dimethyl-3,4-pyrroledicarboxylic acid, diethyl 2,5-dimethyl-3,4-pyrroledicarboxylate, 1,2,5-trimethyl-3,4-pyrroledicarboxylic acid, diethyl 1,2,5-trimethyl-3,4-pyrroledicarboxylate, 1-methyl-2,5-pyrrolediacetic acid, dimethyl 1-methyl-2,5-pyrrolediacetate, dimethyl 3,3′-(2,5-pyrrole)dipropionate, 3,3′-(1-methyl-2,5-pyrrole)dipropionic acid, dimethyl 3,3′-(1-methyl-2,5-pyrrole)dipropionate, diethyl 3,3′-(1-phenyl-2,5-pyrrole)dipropionate, diethyl 1-methyl-2,5-cis-pyrrolidinedicarboxylate, diethyl 1-phenyl-2,5-cis-pyrrolidinedicarboxylate, diethyl 1-methyl-2,5-pyrrolidinediacetate, 3,3′-(1-methyl-2,5-pyrrolidine)dipropionic acid, diethyl 3,3′-(1-methyl-2,5-pyrrolidine)dipropionate, diethyl 2,5-indoledicarboxylate, 2,6-indoledicarboxylic acid, diethyl 2,6-indoledicarboxylate, 9-methyl-1,8-carbazoledicarboxylic acid, 2,6-carbazoledicarboxylic acid, diethyl 2,6-carbazoledicarboxylate, 3,6-carbazoledicarboxylic acid, diethyl 3,6-carbazoledicarboxylate, 9-methyl-3,6-carbazoledicarboxylic acid, diethyl 9-methyl-3,6-carbazoledicarboxylate, 3,4-pyrazoledicarboxylic acid, dimethyl 3,4-pyrazoledicarboxylate, 2-methyl-3,4-pyrazoledicarboxylic acid, 1-phenyl-3,4-pyrazoledicarboxylic acid, dimethyl 1-phenyl-3,4-pyrazoledicarboxylate, 2-phenyl-3,4-pyrazoledicarboxylic acid, dimethyl 2-phenyl-3,4-pyrazoledicarboxylate, 3,5-pyrazoledicarboxylic acid, dimethyl 3,5-pyrazoledicarboxylate, 1-methyl-3,5-pyrazoledicarboxylic acid, dimethyl 1-methyl-3,5-pyrazoledicarboxylate, 1-phenyl-3,5-pyrazoledicarboxylic acid, dimethyl 1-phenyl-3,5-pyrazoledicarboxylate, 4,5-imidazoledicarboxylic acid, diphenyl 4,5-imidazoledicarboxylate, 1-methyl-4,5-imidazoledicarboxylic acid, dimethyl 1-methyl-4,5-imidazoledicarboxylate, 1-phenyl-4,5-imidazoledicarboxylic acid, diethyl 1-phenyl-4,5-imidazoledicarboxylate, 2,3-pyridinedicarboxylic acid, dimethyl 2,3-pyridinedicarboxylate, diphenyl 2,3-pyridinedicarboxylate, 2,4-pyridinedicarboxylic acid, dimethyl 2,4-pyridinedicarboxylate, diphenyl 2,4-pyridinedicarboxylate, 2,5-pyridinedicarboxylic acid, dimethyl 2,5-pyridinedicarboxylate, diphenyl 2,5-pyridinedicarboxylate, 2,6-pyridinedicarboxylic acid, dimethyl 2,6-pyridinedicarboxylate, diphenyl 2,6-pyridinedicarboxylate, 3,4-pyridinedicarboxylic acid, dimethyl 3,4-pyridinedicarboxylate, 3,5-pyridinedicarboxylic acid, diphenyl 3,5-pyridinedicarboxylate, 2,6-dimethyl-3,5-pyridinedicarboxylic acid, 2,4,6-trimethyl-3,5-pyridinedicarboxylic acid, dimethyl 2,5-piperidinedicarboxylate, diethyl 2,3-piperidinedicarboxylate, 2,6-cis-piperidinedicarboxylic acid, dimethyl 2,6-cis-piperidinedicarboxylate, 1-methyl-2,6-cis-piperidinedicarboxylic acid, dimethyl 1-methyl-2,6-cis-piperidinedicarboxylate, diethyl 3,5-piperidinedicarboxylate, 2,6-cis-piperidinediacetic acid, 1-methyl-2,6-cis-piperidinediacetic acid, diethyl 1-methyl-2,6-cis-piperidinediacetate, 2,3-quinolinedicarboxylic acid, dimethyl 2,3-quinolinedicarboxylate, 2,4-quinolinedicarboxylic acid, dimethyl 2,4-quinolinedicarboxylate, 2,6-quinolinedicarboxylic acid, 3,7-quinolinedicarboxylic acid, 4,8-quinolinedicarboxylic acid, dimethyl 4,8-quinolinedicarboxylate, 5,6-quinolinedicarboxylic acid, dimethyl 5,6-quinolinedicarboxylate, 5,8-quinolinedicarboxylic acid, 6,7-quinolinedicarboxylic acid, dimethyl 6,7-quinolinedicarboxylate, 6,8-quinolinedicarboxylic acid, 7,8-quinolinedicarboxylic acid, 2,2′-bipyridine-4,4′-dicarboxylic acid, dimethyl 2,2′-bipyridine-4,4′-dicarboxylate, 2,2′-bipyridine-5,5′-dicarboxylic acid, dimethyl 2,2′-bipyridine-5,5′-dicarboxylate, 2,2′-bipyridine-6,6′-dicarboxylic acid, dimethyl 3,3′-bipyridine-2,2′-dicarboxylate, 4,5-pyridazinedicarboxylic acid, 4,5-pyrimidinedicarboxylic acid, 4,6-pyrimidinedicarboxylic acid, 2,3-pyrazinedicarboxylic acid, dimethyl 2,3-pyrazinedicarboxylate, 2,5-pyrazinedicarboxylic acid, dimethyl 2,5-pyrazinedicarboxylate, diphenyl 2,5-pyrazinedicarboxylate, 2,6-pyrazinedicarboxylic acid, dimethyl 2,6-pyrazinedicarboxylate, dimethyl 1,4-piperazinediacetate, dimethyl 3,3′-(1,4-piperazine)dipropionate, 1,6-phenazinedicarboxylic acid, and dimethyl 1,6-phenazinedicarboxylate. Among these, total aromatic polycarboxylic acids having a rigid molecular structure which does not contain an alkylene chain in the main chain, may be mentioned, and examples include phthalic acids, terephthalic acids, isophthalic acids, biphenyldicarboxylic acids, naphthalenedicarboxylic acids, oxofluorenedicarboxylic acids, anthracenedicarboxylic acids, anthraquinonedicarboxylic acids, biphenylenedicarboxylic acids, terphenyldicarboxylic acids, quaterphenyldicarboxylic acids, azobenzenedicarboxylic acids, furandicarboxylic acids, thiophenedicarboxylic acids, pyranedicarboxylic acids, dibenzofurandicarboxylic acids, dibenzothiophenedicarboxylic acids, xanthenedicarboxylic acids, dibenzo[1,4]dioxindicarboxylic acids, phenoxathiindicarboxylic acids, thianthrenedicarboxylic acids, phenoxaphosphinedicarboxylic acids, pyrroledicarboxylic acids, indoledicarboxylic acids, carbazoledicarboxylic acids, pyrazoledicarboxylic acids, imidazoledicarboxylic acids, pyridinedicarboxylic acids, quinolinedicarboxylic acids, bipyridinedicarboxylic acids, pyrimidinedicarboxylic acids, pyrazinedicarboxylic acids, and phenazinedicarboxylic acids. Furthermore, acid halides or acid anhydrides of the aforementioned aromatic dicarboxylic acids can also be used. Examples of the acid halides of the aromatic dicarboxylic acids include phthaloyl dichloride, and naphthoyl dichloride. Among these, phthaloyl dichloride is preferred, and for example, isophthaloyl dichloride or terephthaloyl dichloride are used.

Preferred terminal protecting agents are disclosed in Japanese Patent Application Laid-Open No. 2007-320989. For example, those represented by the formula: X—C(O)—R may be used, where X is a halogen atom, preferably chlorine, bromine or iodine atom, and more preferably chlorine or bromine atom; R may be a straight or branched alkyl group having 1 to 22 carbon atoms, preferably a straight or branched alkyl group having 1 to 10 carbon atoms, and more preferably a straight or branched alkyl group having 1 to 8 carbon atoms. Examples of the alkyl group include a methyl group, an ethyl group, a butyl group, an octyl group, a dodecyl group, and a t-butyl group. R may be an aryl group having 6 to 30 carbon atoms, preferably having 6 to 24 carbon atoms, more preferably having 6 to 18 carbon atoms. Examples of the aryl group include a phenyl group, groups of fused rings, such as a naphthyl group, an anthranyl group, a fluorenyl group, a phenanthrenyl group, and a pyrenyl group, or groups of rings linked by a carbon-carbon bond, such as a biphenyl group and a terphenyl group. R may be an alkaryl or aralkyl group, which is composed of any alkyl group and an aryl group described above, for example, a benzyl group, a tolyl group, a xylyl group, a butylphenyl group, and a dodecylphenyl group. Furthermore, R may be a group in which at least one of the hydrogen atoms of an alkyl group, aryl group, alkaryl group and aralkyl group is substituted by a fluorine, chlorine, bromine, or iodine atom, or an alkoxyl (—OR), mercapto (—SH), sulfenato (—SO—R), sulfinato (—OSO—R), sulfo (—SO₂OH), alkoxycarbonyl (—COOR), acyl (—CO—R), alkoxysulfinyl (—SO—OR), alkyl-thiocarbonyl (—CO—SR), thiosulfo (—SO₂OR), cyano (—CN), thiocyano (—S—CN), isocyano (—NC), isocyanato (—N═C═O), isothiocyanato (—N═C═S), or nitro (—NO₂) group. This invention, however, is not limited thereto. The alkyl group contained in an alkoxy, alkoxycarbonyl, acyl, alkoxysulfinyl or alkyl-thiocarbonyl group has preferably 1 to 8, more preferably 1 to 4, and most preferably 1 or 2 carbon atoms.

The aromatic polyester resin composition of the present invention comprises the aromatic polyester resin comprising the polyhydric phenol residues, and the residues of the aromatic polycarboxylic acid, acid halide or acid anhydride thereof, and the modifier for aromatic polyesters of the present invention. The upper limit of the content of the modifier for the aromatic polyesters is desirably 50 parts by weight, preferably 40 parts by weight, more preferably 30 parts by weight, and most preferably 20 parts by weight based on 100 parts by weight of the aromatic polyester, while the lower limit is desirably 1 part by weight, preferably 5 parts by weight, more preferably 7 parts by weight, and most preferably 10 parts by weight. A content of the modifier in the polyester exceeding the upper limit may impair the transparency of the aromatic polyester resin composition, while a content below the lower limit may not enhance the fluidity sufficiently. The aromatic polyester composition of the present invention is useful for optical applications such as lenses, optical elements, display substrates and optical fibers, and especially suitable for use in optical fibers.

The present invention is described by way of the following examples in more detail, but should not be limited thereto.

### EXAMPLES

### Modifier for Aromatic Polyesters

Modifiers for aromatic polyesters used in the Examples were prepared in Preparation Examples 1 to 5.

### Preparation Example 1

In a round glass flask were placed 500 ml of water, 22.1 g (0.55 mol) of sodium hydroxide, and 86.2 g (0.525 mol) of 6-t-butyl-2-methyl phenol, and the mixture was stirred at room temperature in a nitrogen atmosphere for 30 minutes to give a homogeneous solution. To the solution was added 403 mg (1.25 mmol) of tetra-n-butylammonium bromide and the solution was stirred at room temperature for 10 minutes in a similar manner.

Subsequently, to the solution was added dropwise 50.5 g (0.25 mol) of phthaloyl dichloride in 400 ml of dichloromethane over a period of 7 minutes, and the mixture was further stirred for 1 hour to complete the esterification. The resulting reaction mixture was transferred to a separatory funnel and the aqueous phase was removed. The organic phase was washed twice each with 200 ml of 0.1 N aqueous sodium hydroxide solution and then was washed three times each with 200 ml of water. Then, the resulting solution was dried sufficiently over about 50 g of anhydrous sodium sulfate. After drying, the anhydrous sodium sulfate was removed by filtration, 250 ml of 95% ethanol was added to the filtrate, and the dichloromethane was distilled off using a rotary evaporator at an atmospheric pressure. Then, the resulting solution was allowed to stand at room temperature to give precipitated white crystals, and the white crystals were recovered by vacuum filtration. The resulting white crude crystals were dissolved in 500 ml of dichloromethane, followed by addition of 250 ml of 95% ethanol, and recrystallization in the same manner. The recovered white crystals weighed 86.0 g (yield: 75%) in total. The white crystals obtained were identified as bis(6-t-butyl-2-methylphenyl)phthalate (Modifier 1), having a melting point of 156° C. to 157° C. measured by a process using a capillary tube for melting point measurement and silicone oil as a heating medium.

### Preparation Example 2

The procedures of Preparation Example 1 were repeated, except that phthaloyl dichloride was replaced with isophthaloyl dichloride, and 6-t-butyl-2-methylphenol (86.2 g, 0.525 mol) was replaced with 4-t-butylphenol (78.9 g, 0.525 mol). The recovered white crystals weighed 102.3 g (yield: 95%) in total. The white crystals obtained were identified as bis(4-t-butylphenyl) isophthalate (Modifier 2), having a melting point of 159° C. to 160° C.

### Preparation Example 3

The procedures of Preparation Example 1 were repeated, except that phthaloyl dichloride was replaced with terephthaloyl dichloride, and 6-t-butyl-2-methylphenol (86.2 g, 0.525 mol) was replaced with 4-t-butylphenol (78.9 g, 0.525 mol). The recovered white crystals weighed 106.5 g (yield: 99%) in total. The white crystals obtained were identified as bis(4-t-butylphenyl) terephthalate (Modifier 3), having a melting point of 216° C. to 220° C.

### Preparation Example 4

The procedures of Preparation Example 1 were repeated, except that 6-t-butyl-2-methylphenol (86.2 g, 0.525 mol) was replaced with 2,2-bis(4′-hydroxyphenyl)propane (bisphenol A) (57.1 g, 0.25 mol), and phthaloyl dichloride (50.5 g, 0.25 mmol) was replaced with 4-t-butylbenzoyl chloride (103.3 g, 0.525 mol). The recovered white crystals weighed 133.0 g (yield: 97%) in total. The white crystals obtained were identified as 4,4′-isopropylidene diphenol di-4-t-butylbenzoate (Modifier 4), having a melting point of 166° C. to 168° C.

### Preparation Example 5

The procedures of Preparation Example 1 were repeated, except that 6-t-butyl-2-methylphenol (86.2 g, 0.525 mol) was replaced with 2,2-bis(4′-hydroxyphenyl)propane[bisphenol A] (57.1 g, 0.25 mol), and phthaloyl dichloride (50.5 g, 0.25 mmol) was replaced with pivaloyl chloride (63.3 g, 0.525 mol). The recovered white crystals weighed 94.1 g (yield: 95%) in total. The white crystals obtained were identified as 4,4′-isopropylidene diphenol di-2,2′-dimethylpropionate (Modifier 5), having a melting point of 123° C. to 125° C.

### Aromatic Polyesters

Properties of aromatic polyesters and compositions thereof were measured in the following manners.

### Glass Transition Temperature (Tg, ° C.)

A differential scanning calorimeter (DSC-3100S, manufactured by Bruker AXS Inc.) was used for measurement. Samples used for measurement were in the form of pellets. The measurement was conducted in a nitrogen atmosphere.

Melt Flow Rate (MFR, g/10 min)

MELT INDEXER F-W01 (trade name, manufactured by Toyo Seiki Seisaku-Sho, Ltd.) was used for measurement. The measurements were conducted at a temperature of 260° C. under a load of 10.0 Kg.

### X-Ray Diffractometry

An X-ray diffractometer RINT2000 (trade name, manufactured by Rigaku Corporation) was used for measurement of melt-extruded samples (melt-extrusion conditions: 260° C., load: 10.0 Kg).

### Preparation of Aromatic Polyesters

A conical glass flask was charged with 1,000 ml of dichloromethane. Then, 2.98 g (21.2 mmol) of benzoyl chloride, 15.37 g (75.7 mmol) of isophthaloyl dichloride and 15.37 g (75.7 mmol) of terephthaloyl dichloride were added and dissolved with sufficient stirring.

Meanwhile, a round bottom glass flask was charged with 1,100 ml of water. Then 13.30 g (0.33 mol) of sodium hydroxide, and 38.0 g (166.6 mmol) of 2,2-bis(4′-hydroxyphenyl)propane[bisphenol A], were added and dissolved with stirring at room temperature for 30 minutes in a nitrogen atmosphere. Then, 12.2 mg (0.038 mmol) of tetra-n-butylammonium bromide was added as a catalyst and stirred at room temperature for 30 minutes in a similar manner.

Subsequently, the entire dichloromethane solution in the conical glass flask was added to the round bottom flask, and the mixture was stirred at room temperature for 30 minutes. After the completion of the reaction, the aqueous phase was removed and the organic phase was transferred to a separatory funnel. Then, 1,000 ml of water was added to the organic phase, and the funnel was shaken and then was allowed to stand to separate the organic phase. The washing operation was repeated three times. The resulting organic phase was added dropwise to a mixed solvent of methanol and water (9:1 v/v) with stirring. The precipitated polymer was collected and dried.

The polymer had a weight average molecular weight (Mw) of 33,000, a glass transition temperature (Tg) of 193° C., and a melt flow rate (MFR) of 1.8 g/10 min.

### Examples 1-5

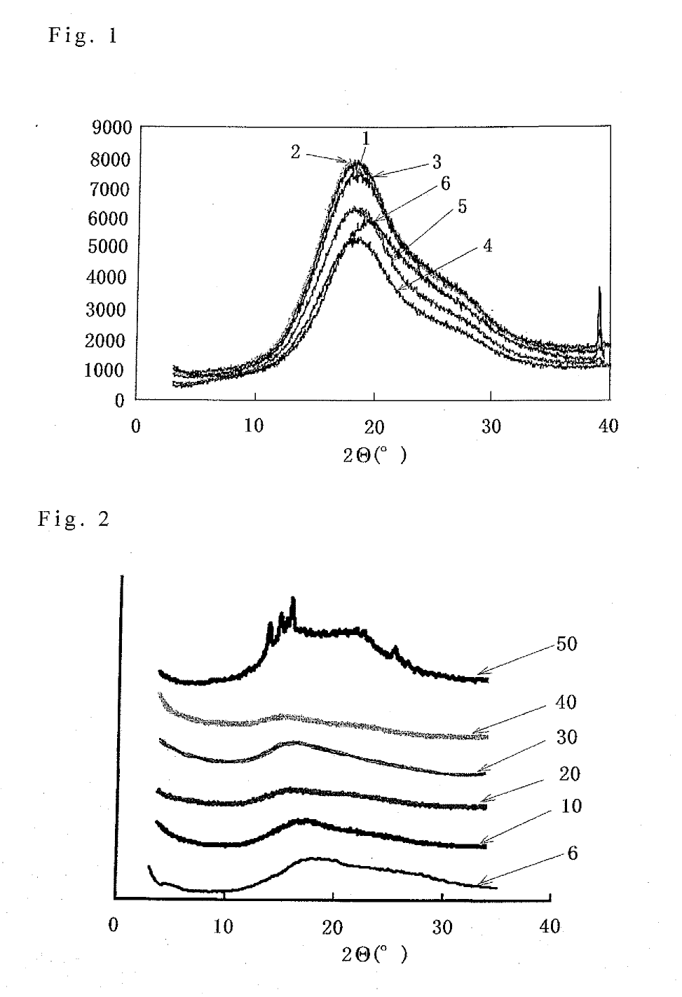

Each of Modifiers 1 to 5 was added independently to the resulting aromatic polyester in an amount of 10 wt %. Glass transition temperatures, melt flow rates, and x-ray diffraction are measured for the aromatic polyester resin compositions, and the glass transition temperatures and the melt flow rates are shown in Table 1, and the results of the x-ray diffractometry are shown in FIG. 1.

TABLE 1

| | Compounding | | | Grass | |
|---|---|---|---|---|---|
| | Ratio (wt %) | | | Transition | Melt Flow |
| | Aromatic | | | Temperature | Rate (MFR, |
| | polyester | Modifier | Modifier | (Tg, ° C.) | g/10 min) |
| Ex. 1 | 90 | 10 | Modifier 1 | 161 | 8.5 |
| Ex. 2 | 90 | 10 | Modifier 2 | 185 | 8.6 |
| Ex. 3 | 90 | 10 | Modifier 3 | 157 | 30.2 |
| Ex. 4 | 90 | 10 | Modifier 4 | 178 | 5.8 |
| Ex. 5 | 90 | 10 | Modifier 5 | 183 | 5.2 |
| Comp. | 100 | 0 | — | 193 | 1.8 |
| Ex. 1 | | | | | |

Ex = Example,
Comp. Ex = Comparative Example

Examples 1 to 3 used bis(6-t-butyl-2-methylphenyl) phthalate, bis(4-t-butylphenyl)isophthalate and bis(4-t-butylphenyl)terephthalate, respectively, as modifiers. Compared with the aromatic polyester of Comparative Example 1 containing no modifier, all the aromatic polyester resin compositions of Examples 1 to 3 exhibited significantly increased melt flow rates and a slightly lower glass transition temperature, which, however, did not have any adverse effect on, for example, the use in optical applications. Examples 4 and 5 used 4,4′-isopropylidene diphenol di-4-t-butylbenzoate, and 4,4′-isopropylidene diphenol di-2,2′-dimethylpropionate, respectively, as modifiers. All the aromatic polyester resin compositions of Examples 4 and 5 containing these modifiers exhibited significantly increased melt flow rates, with slightly lower glass transition temperatures, compared to the aromatic polyester of Comparative Example 1 containing no modifiers. In contrast, the melt flow rate of the aromatic polyester of Comparative Example 1 is lower than those of aromatic polyester resin compositions of Examples 1 to 5; thus, it must be heated to a higher temperature during molding processes compared to the inventive aromatic polyester resin compositions, resulting in slight coloration of the mold product, which was not observed in Examples 1 to 5.

FIG. 1 is an X-ray diffraction chart of the aromatic polyester resin compositions of Examples 1 to 5, and the aromatic polyester resin of Comparative Example 1. FIG. 1 shows that aromatic polyester resin compositions compounded with modifiers 1 to 5 of Examples 1 to 5 were more amorphous than the aromatic polyester containing no modifier in Comparative Example 1. FIG. 2 is an X-ray diffraction chart of the aromatic polyester resin compositions containing varying amounts of modifier 1 in an amount of 10 to 50 parts by weight based on 100 parts by weight of the polyesters, and of the aromatic polyester of Comparative Example 1. FIG. 2 shows that an increased amount of modifier 1 leads to an increase in the degree of amorphousity. Furthermore, addition of 50 parts by weight of modifier 1 leads to a slight increase in crystallinity. The amount of modifier 1 may preferably be no greater than 50 parts by weight since the transparency decreases with an increase in crystallinity.

The modifier for aromatic polyesters of the present invention may enhance the melt fluidity of aromatic polyesters without a significant decrease in the heat resistance of the aromatic polyesters. Accordingly, the aromatic polyester resin compositions containing the modifiers for aromatic polyesters of the present invention are barely colored during molding processes. Accordingly, the modifiers for aromatic polyesters and the aromatic polyester resin compositions including the modifiers are useful for optical applications such as lenses, optical elements, display substrates and optical fibers, and especially suitable for use in optical fibers.

1. A modifier for aromatic polyesters comprising polyhydric phenol residues and residues of aromatic polycarboxylic acid, acid halide or acid anhydride thereof;
wherein the modifier comprises a material having a structure composed of a first residue selected from the group consisting of divalent residues represented by Formula (I):
—Ar—W¹ₓ—Ar— (I)
where each Ar independently denotes a phenylene or naphthylene group, and may be independently substituted by at least one group and/or atom selected from the group consisting of a saturated or unsaturated acyclic or cyclic alkyl group having 1 to 12 carbon atoms, a saturated or unsaturated acyclic or cyclic alkoxy group having 1 to 12 carbon atoms, a halogen atom, a nitro group, an —SR¹ group (where R¹ denotes a hydrogen atom or an alkyl group having 1 to 4 carbon atoms), and an —R³—NR²₂ group (where each R² independently denotes a hydrogen atom or an alkyl group having 1 to 4 carbon atoms, and R³ denotes an alkylene group having 1 to 4 carbon atoms or may be absent), and x is 0 or 1, where when x is 1, W¹ denotes a saturated or unsaturated acyclic or cyclic alkylene group having 1 to 30 carbon atoms, an oxygen atom, a sulfur atom, sulfonyl group, or sulfonyl group, and when x is 0, two Ar's are covalently bonded by a carbon atom contained in each Ar;
and represented by Formula (II):
—Ar— (II)
where Ar denotes a phenylene group or naphthylene group, and Ar may be substituted by at least one group and/or atom selected from the group consisting of a saturated or unsaturated acyclic or cyclic alkyl group having 1 to 12 carbon atoms, a saturated or unsaturated acyclic or cyclic alkoxy group having 1 to 12 carbon atoms, a halogen atom, a nitro group, an —SR¹ group (where R¹ denotes a hydrogen atom or an alkyl group having 1 to 4 carbon atoms), a —R³—NR²₂ group (where each R² independently denotes a hydrogen atom or an alkyl group having 1 to 4 carbon atoms, and R³ denotes an alkylene group having 1 to 4 carbon atoms or may be absent), and a phenyl group optionally substituted by an alkyl group having 1 to 4 carbon atoms;
the first residue being bonded to two identical or different second residues selected from the group consisting of monovalent residues represented by Formula (III): 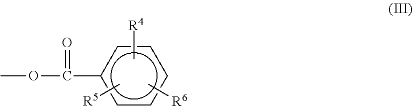
where R⁴, R⁵, and R⁶ each independently denotes a hydrogen atom or a saturated or unsaturated acyclic alkyl group having 1 to 5 carbon atoms;
and represented by Formula (IV):
—O—C(O)—R⁷— (IV)
where R⁷ denotes a saturated or unsaturated acyclic alkyl group having 1 to 5 carbon atoms.

2. The modifier for aromatic polyesters according to claim 1, wherein, in Formula (I), each Ar independently denotes a phenylene or naphthylene group, and may be independently substituted by at least one group and/or atom selected from the group consisting of a saturated or unsaturated acyclic or cyclic alkyl group having 1 to 6 carbon atoms, a saturated or unsaturated acyclic or cyclic alkoxy group having 1 to 6 carbon atoms, a halogen atom, a nitro group, an —SR¹ group (where R¹ denotes a hydrogen atom or an alkyl group having 1 to 4 carbon atoms), and an —R³—NR²₂ group (where each R² independently denotes a hydrogen atom or an alkyl group having 1 to 4 carbon atoms, and R³ denotes an alkylene group having 1 to 4 carbon atoms or may be absent), and x is 0 or 1, where when x is 1, W¹ denotes a saturated or unsaturated acyclic or cyclic alkylene group having 1 to 6 carbon atoms, an oxygen atom, a sulfur atom, a sulfinyl group, or a sulfonyl group, and when x is 0, two Ar's are covalently bonded by a carbon atom contained in each Ar.

3. The modifier for aromatic polyesters according to claim 1, wherein, in Formula (I), each Ar independently denotes a phenylene or naphthylene group, and may be independently substituted by at least one group and/or atom selected from the group consisting of a saturated acyclic alkyl group having 1 to 4 carbon atoms, a saturated acyclic alkoxy group having 1 to 4 carbon atoms, a halogen atom, a nitro group, an —SR¹ group (where R¹ denotes a hydrogen atom or an alkyl group having 1 to 4 carbon atoms), and an —R³—NR²₂ group (where each R² independently denotes a hydrogen atom or an alkyl group having 1 to 4 carbon atoms; and R³ denotes an alkylene group having 1 to 4 carbon atoms or may be absent), and x is 0 or 1, where when x is 1, W¹ denotes a saturated acyclic alkylene group having 1 to 4 carbon atoms, an oxygen atom, a sulfur atom, a sulfinyl group, or a sulfonyl group, and when x is 0, two Ar's are covalently bonded by a carbon atom contained in each Ar.

4. The modifier for aromatic polyesters according to claim 1, wherein, in Formula (I), each Ar independently denotes a phenylene or naphthylene group, and may be independently substituted by at least one group and/or atom selected from the group consisting of a saturated acyclic alkyl group having 1 to 4 carbon atoms, and a halogen atom, x is 1, and W¹ denotes a saturated acyclic alkylene group having 1 to 4 carbon atoms.

5. The modifier for aromatic polyesters according to claim 1, wherein, in Formula (I), each Ar denotes a phenylene group, and may be independently substituted by a saturated acyclic alkyl group having 1 to 4 carbon atoms, x is 1, and W¹ denotes a saturated acyclic alkylene group having 1 to 4 carbon atoms.

6. The modifier for aromatic polyesters according to claim 1, in Formula (I), each Ar denotes a phenylene group, x is 1, and W¹ denotes a saturated acyclic alkylene group having 1 to 3 carbon atoms.

7. The modifier for aromatic polyesters according to claim 1, wherein, in Formula (II), Ar denotes a phenylene group or a naphthylene group, and Ar may be substituted by at least one group and/or atom selected from the group consisting of a saturated or unsaturated acyclic or cyclic alkyl group having 1 to 6 carbon atoms, a saturated or unsaturated acyclic or cyclic alkoxy group having 1 to 6 carbon atoms, a halogen atom, a nitro group, an —SR¹ group (where R¹ denotes a hydrogen atom or an alkyl group having 1 to 4 carbon atoms), a —R³—NR²₂ group (where each R² independently denotes a hydrogen atom or an alkyl group having 1 to 4 carbon atoms, and R³ denotes an alkylene group having 1 to 4 carbon atoms or may be absent), and a phenyl group optionally substituted by an alkyl group having 1 to 4 carbon atoms.

8. The modifier for aromatic polyesters according to claim 1, wherein, in Formula (II), Ar denotes a phenylene group or a naphthylene group, and Ar may be substituted by at least one group and/or atom selected from the group consisting of a saturated acyclic alkyl group having 1 to 4 carbon atoms, a saturated acyclic alkoxy group having 1 to 4 carbon atoms, a halogen atom, a nitro group, an —SR¹ group (where R¹ denotes a hydrogen atom or an alkyl group having 1 to 4 carbon atoms), a —R³—NR²₂ group (where each R² independently denotes a hydrogen atom or an alkyl group having 1 to 4 carbon atoms, and R³ denotes an alkylene group having 1 to 4 carbon atoms or may be absent), and a phenyl group optionally substituted by an alkyl group having 1 to 4 carbon atoms.

9. The modifier for aromatic polyesters according to claim 1, wherein, in Formula (II), Ar denotes a phenylene group or a naphthylene group, and may be substituted by at least one group and/or atom selected from the group consisting of a saturated acyclic alkyl group having 1 to 4 carbon atoms and a halogen atom.

10. The modifier for aromatic polyesters according to claim 1, wherein, in Formula (II), Ar denotes a phenylene group, and may be substituted by a saturated acyclic alkyl group having 1 to 4 carbon atoms.

11. The modifier for aromatic polyesters according to claim 1, wherein, in Formula (II), Ar denotes a phenylene group.

12. The modifier for aromatic polyesters according to claim 1, wherein, in Formula (III), R⁴, R⁵, and R⁶ each independently denotes a hydrogen atom, or a saturated acyclic alkyl group having 1 to 5 carbon atoms.

13. The modifier for aromatic polyesters according to claim 1, wherein, in Formula (III), any one of R⁴, R⁵, and R⁶ denotes a hydrogen atom, and each of the other two groups independently denotes a saturated acyclic alkyl group having 1 to 5 carbon atoms.

14. The modifier for aromatic polyesters according to claim 1, wherein, in Formula (IV), R⁷ denotes a saturated acyclic alkyl group having 1 to 5 carbon atoms.

15. The modifier for aromatic polyesters according to claim 1, being selected from the group consisting of diphenyl phthalate, diphenyl isophthalate, diphenyl terephthalate, bis(4-t-butylphenyl)phthalate, bis(4-t-butylphenyl)isophthalate, bis(4-t-butylphenyl)terephthalate, bis(6-t-butyl-2-methylphenyl)phthalate, bis(2-methyl-6-t-butylphenyl)isophthalate, bis(2-methyl-6-t-butylphenyl)terephthalate, 4,4′-isopropylidenediphenol di-4-t-butylbenzoate, 4,4′-isopropylidenediphenol di-2-t-butylbenzoate, and 4,4′-isopropylidenediphenol di-2,2′-dimethylpropionate.

16. The modifier for aromatic polyesters according to claim 1, being selected from the group consisting of diphenyl phthalate, diphenyl isophthalate, diphenyl terephthalate, bis(4-t-butylphenyl)phthalate, bis(4-t-butylphenyl)isophthalate, bis(4-t-butylphenyl)terephthalate, bis(6-t-butyl-2-methylphenyl)phthalate, bis(2-methyl-6-t-butylphenyl)isophthalate, bis(2-methyl-6-t-butylphenyl)terephthalate, 4,4′-isopropylidenediphenol di-4-t-butylbenzoate, and 4,4′-isopropylidenediphenol di-2,2′-dimethylpropionate.

17. The modifier for aromatic polyesters according to claim 1, being selected from the group consisting of bis(4-t-butylphenyl)phthalate, bis(4-t-butylphenyl)isophthalate, bis(4-t-butylphenyl)terephthalate, bis(6-t-butyl-2-methylphenyl)phthalate, bis(2-methyl-6-t-butylphenyl)isophthalate, bis(2-methyl-6-t-butylphenyl)terephthalate, 4,4′-isopropylidenediphenol di-4-t-butylbenzoate, and 4,4′-isopropylidenediphenol di-2,2′-dimethylpropionate.

18. An aromatic polyester resin composition comprising the modifier for aromatic polyesters according to claim 1.

19. An aromatic polyester resin composition comprising the modifier for aromatic polyesters according to claim 1 in an amount of 1 to 50 parts by weight based on 100 parts by weight of the aromatic polyesters.

20. An aromatic polyester resin composition comprising the modifier for aromatic polyesters according to claim 1 in an amount of 5 to 40 parts by weight based on 100 parts by weight of the aromatic polyesters.
